(19) United States
(12) Patent Application Publication
(10) Pub. No.: US 2024/0207266 A1
(43) Pub. Date: Jun. 27, 2024

(54) METHODS FOR TREATING AND MONITORING PARKINSON'S DISEASE

(71) Applicant: Denali Therapeutics Inc., South San Francisco, CA (US)

(72) Inventors: Danna L. Jennings, Half Moon Bay, CA (US); Vinay M. Daryani, South San Francisco, CA (US); Sarah Huntwork-Rodriguez, South San Francisco, CA (US)

(21) Appl. No.: 18/288,734

(22) PCT Filed: Apr. 29, 2022
(86) PCT No.: PCT/US2022/026898

§ 371 Date: Oct. 27, 2023

Related U.S. Application Data
(60) Provisional application No. 63/182,207, filed on Apr. 30, 2021.

(51) Int. Cl. A61K 31/506; A61K 9/00; A61K 9/20; A61K 9/28

(52) U.S. Cl. CPC A61K 31/506; A61K 9/0053; A61K 9/2009; A61K 9/2013; A61K 9/2027; A61K 9/2054; A61K 9/284

(57) ABSTRACT

The present disclosure relates to methods for treating Parkinson's disease in a subject with a compound provided herein, pharmaceutical compositions comprising the compound, as well methods for monitoring subject's response to the treatment.

### CROSS REFERENCE TO RELATED APPLICATION

This application claims the benefit under 35 U.S.C. § 119(e) to U.S. Provisional Application No. 63/182,207 filed Apr. 30, 2021, which is incorporated by reference in its entirety.

### FIELD

The present disclosure relates to methods for treating and/or monitoring Parkinson's disease.

### BACKGROUND

Combined genetic and biochemical evidence implicates certain kinase function in the pathogenesis of neurodegenerative disorders (Christensen, K. V. (2017) Progress in Medicinal Chemistry 56:37-80; Fuji, R. N. et al (2015) Sci. Transl. Med. 7(273):ra15; Taymans, J. M. et al (2016) Curr. Neuropharm. 14(3):214-225). Parkinson's disease is a neurodegenerative disease that affects the neurological system presenting with both motor and non-motor symptoms. Although the exact causes of Parkinson's disease are unknown, it is believed that a combination of genetic and environmental factors contribute to the etiology of the disease. Among the genes that have been implicated in Parkinson's disease is Park8, which encodes the leucine-rich repeat kinase 2 (LRRK2), a complex signaling protein that is a key therapeutic target in Parkinson's disease (PD). Mutations in Park8 are found in both familial and non-familial (sporadic) forms of Parkinson's disease, and increased kinase activity of LRRK2 is implicated in the pathogenesis of Parkinson's disease. Mutations in the LRRK2 gene are the most frequent genetic cause of familial Parkinson's disease and a major driver of lysosomal dysfunction, which contribute to the formation of Parkinson's disease pathogenesis and neurodegeneration. (Chai C, et al. Curr Genomics. 2013:14:464-471; Healy D G, et al. Lancet Neurol. 2008:7:583-590; Henry A G, et al. Human Mol. Gen. 2015:24:6013-6028; Cookson M R, et al. Nat. Rev. Neurosci. 2016; 11:791-797). LRRK2 regulates lysosomal genesis and function, which is impaired in Parkinson's disease and may be restored by LRRK2 inhibition, thereby potentially positively modifying disease progression in patients with a genetic LRRK2 mutation as well as in patients with sporadic Parkinson's disease.

Combined genetic and biochemical evidence supports a model in which the LRRK2 kinase function is causally involved in the pathogenesis of sporadic and familial forms of PD, and therefore that LRRK2 kinase inhibitors appear to be useful for treatment (Christensen, K. V. (2017) Progress in Medicinal Chemistry 56:37-80). Inhibition of the kinase activity of LRRK2 is under investigation as a treatment for Parkinson's disease (Fuji, et al., 2015; Taymans, J. M. et al (2016) Current Neuropharmacology 14(3):214-225).

LRRK2 kinase inhibitors have been studied for treatment of Alzheimer's disease, Parkinson's disease, ALS and other neurodegenerative diseases (Estrada, A. A. et al (2015) Jour. Med Chem. 58(17): 6733-6746; Estrada. A. A. et al (2013) Jour. Med. Chem. 57:921-936; Chen, H. et al (2012) Jour. Med. Chem. 55:5536-5545; Estrada. A. A. et al (2015) Jour. Med. Chem. 58:6733-6746: Chan, B. K. et al (2013) ACS Med. Chem. Lett. 4:85-90; U.S. Pat. Nos. 8,354,420; 8,569,281; 8,791,130; 8,796,296; 8,802,674; 8,809,331; 8,815,882; 9,145,402; 9,212,173; 9,212,186; 9,932,325; WO 2011/151360: WO 2012/062783; WO 2013/079493).

Administration of various LRRK2 kinase inhibitors is known to induce changes in lysosomal morphology and tissue levels of lipids associated with the lysosome. Accordingly, administration of LRRK2 inhibitors GNE-7915 and GNE-0877 in monkeys resulted in decreased urine di-22:6-BMP (Fuji R N, et al (2015) Sci. Transl. Med 7(273):273ra215; Baptista M A, et al Baptista et al., (2020) Sci. Transl. Med. 12(540).

Di-22:6-BMP is a phospholipid that is normally localized in the internal membrane of lysosomes and late endosomes, and is responsible for lysosomal degradation. Enlarged and increased numbers of lysosomes with stacked, whorled membranes and lipid were also observed in proximal tubules of LRRK2 knockout mice kidney (Herzig M C. et al. (2011) Hum. Mol. Genet. 20(21):4209-4223), suggestive of accumulated phospholipid membranes in lysosomes. Drug-induced phospholipidosis (PLD) is an acquired lysosomal storage disorder characterized by excessive accumulation of phospholipids and drugs in lysosomes in different tissues such as kidney, heart, and lungs (Shayman J A, et al (2013) Biochim. Biophys. Acta. 1831(3):602-611; Atashrazm, F. (2016) Clinical Pharmacology: Advances and Applications 8:177-189).

There is a need for methods for treating and/or monitoring the progression of the treatment of Parkinson's disease.

### DESCRIPTION

The following brief summary is not intended to include all features and aspects of the present invention, nor does it imply that the invention must include all features and aspects discussed in this summary.

The present disclosure relates to methods for treating Parkinson's disease, the method comprising administering to a subject in need thereof between about 70 to about 800 mg/day of compound I, N2-(3-(2-(2H-1,2,3-triazol-2-yl)propan-2-yl)-1-cyclopropyl-1H-pyrazol-5-yl)-N4-ethyl-5-(trifluoromethyl)pyrimidine-2,4-diamine:

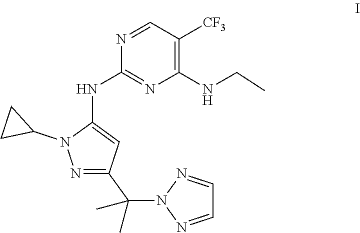 [SMILES: CCNc1nc(Nc2cc(C(C)(C)n3nccn3)nn2C2CC2)ncc1C(F)(F)F]

or a pharmaceutically acceptable salt or deuterated analog thereof.

In another aspect, provided is a method for treating Parkinson's disease, the method comprising administering to a subject in need thereof a pharmaceutical composition comprising between about 70 to about 800 mg/day of compound I:

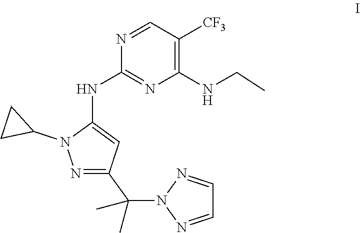 [SMILES: CCNc1nc(Nc2cc(C(C)(C)n3nccn3)nn2C2CC2)ncc1C(F)(F)F]

or a pharmaceutically acceptable salt or deuterated analog thereof and a pharmaceutically acceptable carrier.

In one aspect, the present disclosure provides methods for treating Parkinson's disease with about 70 to about 225 mg/day of compound I or a pharmaceutically acceptable salt or deuterated analog thereof.

In another aspect, the disclosure relates to methods for treating Parkinson's disease with about 70 to about 80 mg/day of compound I or a pharmaceutically acceptable salt or deuterated analog thereof.

In other aspects, about 70 mg, about 75 mg, about 80 mg, about 105 mg, about 130 mg, about 150 mg, about 225 mg, about 250 mg, about 300 mg or about 400 mg is administered to the subject.

In one aspect, compound I or a pharmaceutically acceptable salt or deuterated analog thereof is administered orally.

In one aspect, compound I or a pharmaceutically acceptable salt or deuterated analog thereof is administered once daily.

In another aspect, compound I or a pharmaceutically acceptable salt or deuterated analog thereof is administered twice daily.

In other aspects, the methods provided herein are for treating a human. In still other aspects the methods are for treating familial Parkinson's disease. In yet other aspects the methods are for treating sporadic Parkinson's disease.

In yet another aspect, the method results in a reduction in phosphorylated S935 LRRK2 (pS935) in whole blood of the subject.

In still another aspect, the method results in a reduction in phosphorylated ras-related protein Rab10 (pRab10) in peripheral blood mononuclear cells (PBMC) of the subject.

In yet another aspect, the method results in a reduction of lysosomal lipid 22:6-bis[monoacylglycerol]phosphate (BMP) in urine of the subject.

In another aspect, provides is a method for reducing phosphorylated S935 LRRK2 (pS935) in whole blood of a subject suffering from Parkinson's disease, the method comprising administering to a subject in need thereof between about 70 to 800 mg/day of compound I or a pharmaceutically acceptable salt or deuterated analog thereof.

In one aspect, the pS935 is reduced by at least 41-97%.

In yet another aspect, provided is a method for reducing phosphorylated ras-related protein Rab10 (pRab10) in peripheral blood mononuclear cells (PBMC) of a subject suffering from Parkinson's disease, the method comprising administering to a subject in need thereof between about 70 to 800 mg/day of compound I or a pharmaceutically acceptable salt or deuterated analog thereof.

In one aspect, the pRab10 is reduced by at least 44-97%.

In another aspect, provided is a method for reducing lysosomal lipid 22:6-bis[monoacylglycerol]phosphate (BMP) in urine of a subject suffering from Parkinson's disease, the method comprising administering to a subject in need thereof between about 70 to 800 mg/day of compound I or a pharmaceutically acceptable salt or deuterated analog thereof.

In one aspect, BMP(22:6/22:6) or BMP(22:6/22:6)/creatinine is reduced by 22-86% or by at least 40%.

In another aspect, provided is the use of a LRRK2 inhibitor for treating Parkinson's disease, wherein the inhibitor is administered to a subject in need thereof between about 70 to 800 mg/day and is compound I or a pharmaceutically acceptable salt or deuterated analog thereof.

In one aspect, provided is use of a LRRK2 inhibitor in the manufacture of a medicament for treating Parkinson's disease, wherein the inhibitor is administered to a subject in need thereof between about 70 to 800 mg/day is compound I or a pharmaceutically acceptable salt or deuterated analog thereof.

In another aspect, provided are methods of assessing treatment by detecting a reduction in detecting a reduction in phosphorylated S935 LRRK2 (pS935), phosphorylated ras-related protein Rab10 (pRab10) or lysosomal lipid 22:6-bis[monoacylglycerol]phosphate (BMP) in a patient sample.

In one aspect, provided is a method for monitoring a subject's response to the treatment methods provided herein, the method comprising: (a) measuring an amount of one or more pS935, pRab10 and/or BMP species in a test sample from a subject treated with between about 70 to 800 mg/day of compound I or a pharmaceutically acceptable salt or deuterated analog thereof; (b) comparing the difference in amount between the one or more pS935, pRab10 and/or BMP species measured in (a) and one or more reference values; and (c) determining from the comparison whether the compound, pharmaceutical composition, or dosing regimen thereof improves one or more pS935, pRab10 and/or BMP species levels for treating Parkinson's disease.

In another aspect, the method further comprises altering the dosage or frequency of dosing of compound I or a pharmaceutically acceptable salt or deuterated analog thereof, or the course of therapy administered to the patient.

In yet another aspect, the invention relates to a pharmaceutical composition comprising 70-800 mg of compound I,

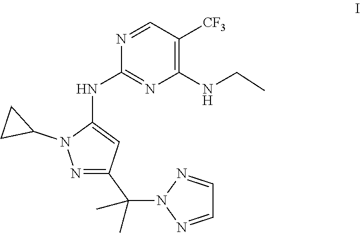 [SMILES: CCNc1nc(Nc2cc(C(C)(C)n3nccn3)nn2C2CC2)ncc1C(F)(F)F]

or a pharmaceutically acceptable salt or deuterated analog thereof and a pharmaceutically acceptable carrier.

In another aspect, the invention relates to a pharmaceutical composition comprising about 70-225 mg of compound I.

In still another aspect, the invention relates to a pharmaceutical composition of compound I, suitable for administration of about 225 mg per day or up to 800 mg per day.

In still other aspects, the invention relates to a pharmaceutical composition comprising about 70 mg, about 75 mg, about 80 mg, about 105 mg, about 130 mg, about 150 mg, about 225 mg, about 250 mg, about 300 mg, or about 400 mg of compound I.

In another aspect, the invention relates to a pharmaceutical composition of compound I, suitable for oral administration.

In other aspects, the invention relates to a pharmaceutical composition of compound I, suitable for administration once, twice or three times daily.

The features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the proposed mechanism of action of LRRK2, comparing a Parkinson's Disease cell to a LRRK2 Inhibitor-treated cell, aSyn=α-synuclein; GBA=β-glucocerobrosidase; LRRK2=leucine-rich repeat kinase 2; Rabs=Rab GTPase.

FIG. 2 shows a Phase 1 study design. This double-blind, placebo-controlled Phase 1 study comprised single-ascending dose (SAD) and 10-day, 14-day, and 28-day multiple-ascending dose (MAD) parts in healthy volunteers. BID=twice daily; PBO=placebo; QD=once daily.

FIG. 3 shows a Phase 1b study design. This study was a double-blind, placebo-controlled, parallel-design Phase 1b study with 28-day dosing, administered once daily in Parkinson's disease patients.

FIGS. 4A and 4B show target engagement in the Phase 1 study. BL=baseline; IQR=interquartile range; MAD=multiple-ascending dose. FIG. 4A shows percent reduction of whole blood pS935 (baseline to day 10). FIG. 4B shows percent reduction of whole blood pS935 (baseline to day 14). Abbreviations: IQR=interquartile range; pS935 LRRK2=leucine-rich repeat kinase 2 serine 935 phosphorylation: QD=once daily; BID—twice daily.

FIGS. 5A and 5B show pathway engagement in the Phase 1 study. FIG. 5A shows percent reduction in pRab10 from PBMCs (baseline to day 10). FIG. 5B shows percent reduction in pRab10 from PBMCs (baseline to day 14).

FIGS. 6A and 6B show target and pathway engagement in the Phase 1b study. FIG. 6A shows percent reduction of whole blood pS935 (baseline to day 28). FIG. 6B shows percent reduction in pRab10 from PBMCs (baseline to day 28).

FIGS. 7A and 7B show lysosomal engagement in Phase 1/1b studies with compound I. FIG. 7A shows percent reduction in BMP(22:6/22:6) (baseline to day 10 [part B], day 28 [part D], and day 14 [part E]) in Phase I healthy volunteers (parts B, D, and E MAD cohorts). FIG. 7B shows percent reduction in urinary BMP(22:6/22:6)/creatinine (baseline to Day 10 [Part B], Day 28 [[Part D], and Day 14 [Part E]) in Phase 1b patients with Parkinson's disease. BMP concentrations were normalized to creatinine concentrations (ng/mg).

FIG. 8 shows the demographics and clinical characteristics of patients with Parkinson's disease in the Phase 1b study. H&Y, Hoehn and Yahr; MDS-UDPRS III, Movement Disorders Society-Unified Parkinson's Disease Rating Scale; MAO-B, monoamine oxidase; PD, Parkinson's disease, QD, once daily.

FIG. 9 shows treatment-emergent adverse events in the MAD cohorts in the Phase 1 study in healthy volunteers. *Procedure related includes (in order of frequency): Procedural pain, procedural headache, post procedural complication, puncture site pain, puncture site puritis, puncture site pain, catheter site pain, post procedural discomfort, medical device dermatitis, catheter site erythema. In a separate analysis of ≥1 TEAE in ≥2 subjects per treatment arm included the following additional TEAEs not listed above: ear pain (n=2; 105 mg QD 10-day cohort); nasopharyngitis (n=2; 225 mg QD 28-day cohort); asymptomatic COVID-19 (n=2; 400 mg BID 14-day cohort); somnolence (n=2; 250 mg BID 14-day cohort). 2 subjects also experienced presyncope associated with lumbar puncture (one each for 150 and 225 mg QD 28-day cohort).

FIG. 10 shows treatment-emergent adverse events in the Phase 1b study in Parkinson's disease patients. GERD, gastroesophageal reflux disease: TEAE, treatment-emergent adverse event. ^{a}Procedural related includes (in order of frequency): procedural pain, post procedural contusion, post procedural hematoma, and procedural headache; ^{b}Hypotension and orthostatic hypotension occurred in the same two patients.

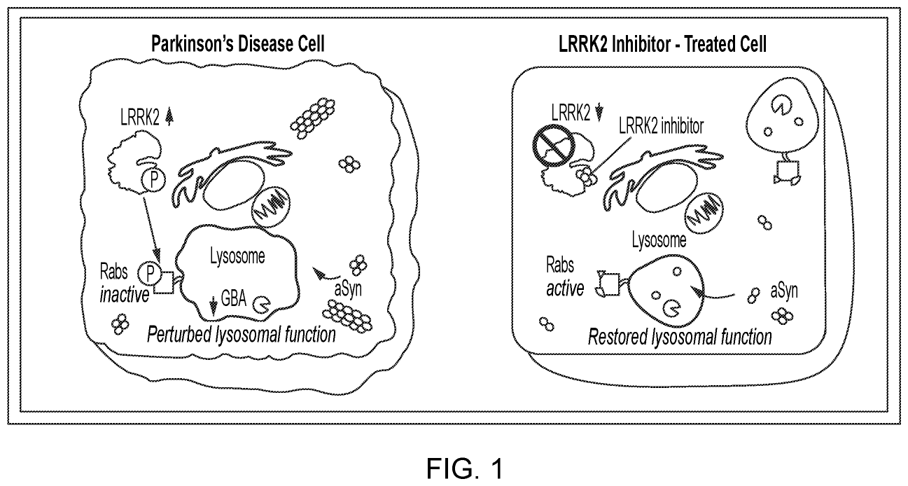

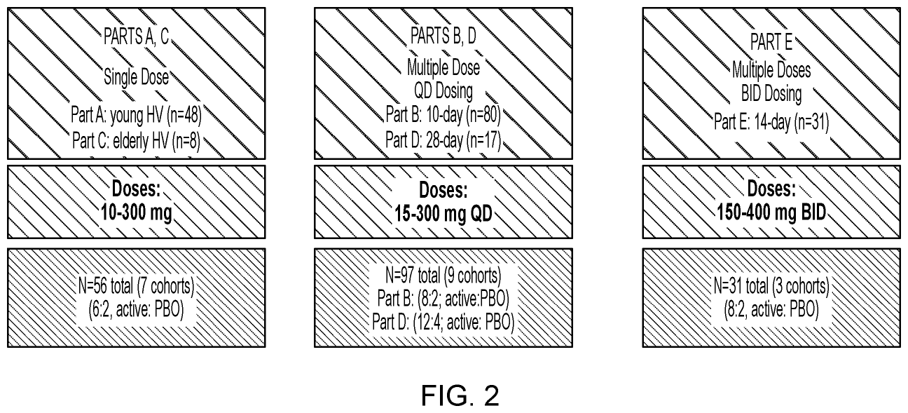

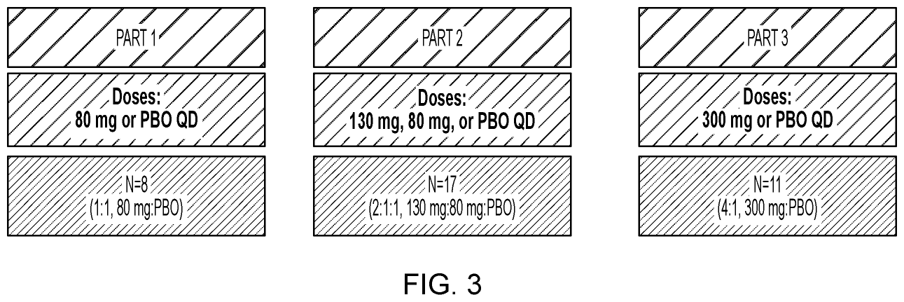

### DEFINITIONS

Unless defined otherwise, all technical and scientific terms used herein have the same meaning as commonly understood by those of ordinary skill in the art to which this invention belongs. Although any methods and materials similar or equivalent to those described herein can be used in the practice or testing of the present invention, the preferred methods and materials are described. Generally, nomenclatures utilized in connection with, and techniques of, cell and molecular biology and chemistry are those well-known and commonly used in the art. Certain experimental techniques, not specifically defined, are generally performed according to conventional methods well known in the art and as described in various general and more specific references that are cited and discussed throughout the present specification. For purposes of clarity, following terms are defined below.

The words “comprise,” “comprising,” “include,” “including,” and “includes” when used in this specification and claims are intended to specify the presence of stated features, integers, components, or steps, but they do not preclude the presence or addition of one or more other features, integers, components, steps, or groups thereof.

The terms “treat” and “treatment” refer to both therapeutic treatment and prophylactic or preventative measures, wherein the object is to prevent or slow down (lessen) an undesired physiological change or disorder, such as the growth, development or spread of a lysosomal dysfunction disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The term “about” indicates that a value includes the inherent variation of error for the method being employed to determine a value, or the variation that exists among experiments. The term “about” may refer to a variation of +/−10%.

The term “amount” refers to the level or concentration of a molecule, compound, or agent (e.g., a pS935, pRab10 or BMP molecule). The term includes an absolute amount or concentration, as well as a relative amount or concentration. In some embodiments, a reference standard (e.g., an internal pS935, pRab10 or BMP standard) is used for calibration in order to determine the absolute amount or concentration of a molecule, compound, or agent that is present (e.g., in a sample) and/or normalize to a control in order to determine a relative amount or concentration of a molecule, compound, or agent that is present.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. Efficacy can be measured, for example, by assessing the time to disease progression (TTP) and/or determining the response rate (RR).

The term “detection” includes any means of detecting, including direct and indirect detection.

“Change” or “modulation” of the status of a biomarker, including a LRRK2 mutation or amount of a BMP, as it occurs in vitro or in vivo is detected by analysis of a biological sample using one or more methods commonly employed in establishing pharmacodynamics, including: (1) sequencing the genomic DNA or reverse-transcribed PCR products of the biological sample, whereby one or more mutations are detected: (2) evaluating gene expression levels by quantitation of message level or assessment of copy number; and (3) analysis of proteins by immunohistochemistry, immunocytochemistry, ELISA, or mass spectrometry whereby degradation, stabilization, or post-translational modifications of the proteins such as phosphorylation or ubiquitination is detected.

The term “subject” includes, but is not limited to, humans, mice, rats, guinea pigs, monkeys, dogs, cats, horses, cows, pigs and sheep. In some embodiments the subject is a human.

The terms “optional” or “optionally” means that the subsequently described event or circumstance may or may not occur and that the description includes instances where said event or circumstance occurs and instances in which it does not.

The term “package insert” is used to refer to instructions customarily included in commercial packages of therapeutic products, that contain information about the indications, usage, dosage, administration, contraindications and/or warnings concerning the use of such therapeutic products.

Any compound or structure given herein, is also intended to represent unlabeled forms as well as isotopically labeled forms of the compounds. Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I and ¹²⁵I, respectively. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of compounds described herein in which from 1 to n hydrogens attached to a carbon atom is/are replaced by deuterium, in which n is the number of hydrogens in the molecule. Such compounds exhibit increased resistance to metabolism and are thus useful for increasing the half-life of any compound when administered to a mammal, particularly a human. See, for example, Foster, “Deuterium Isotope Effects in Studies of Drug Metabolism.” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, ¹¹C labeled compound may be useful for PET or SPECT or other imaging studies. Isotopically labeled compounds of this disclosure can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto.

Provided are also pharmaceutically acceptable salts of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. Other salts include acid salts such as coformers described above. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

The desired pharmaceutically acceptable salt may be prepared by any suitable method available in the art. For example, treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, methanesulfonic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. Acids which are generally considered suitable for the formation of pharmaceutically useful or acceptable salts from basic pharmaceutical compounds are discussed, for example, by Stahl P H, Wermuth C G, editors. Handbook of Pharmaceutical Salts: Properties, Selection and Use, 2^{nd} Revision (International Union of Pure and Applied Chemistry). 2012, New York: Wiley-VCH: S. Berge et al, Journal of Pharmaceutical Sciences (1977) 66(1) 119; P. Gould, International J. of Pharmaceutics (1986) 33 201 217; Anderson et al, The Practice of Medicinal Chemistry (1996). Academic Press, New York: Remington's Pharmaceutical Sciences, 18^{th} ed., (1995) Mack Publishing Co., Easton PA; and in The Orange Book (Food & Drug Administration, Washington, D.C. on their website). These disclosures are incorporated herein by reference thereto.

The phrase “pharmaceutically acceptable” indicates that the substance or composition must be compatible chemically and/or toxicologically, with the other ingredients comprising a formulation, and/or the mammal being treated therewith.

As used herein, “pharmaceutically acceptable carrier” or “pharmaceutically acceptable excipient” or “excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

### Target and Pathway Biomarkers of LRRK2 Activity

Lysosomal dysfunction is a central pathophysiology of Parkinson's Disease (PD) in patients with and without known genetic drivers of PD. Increased LRRK2 kinase activity impairs lysosomal function and drives familial PD. LRRK2 inhibition can restore normal lysosomal function and reduce toxicity in (PD) models. Inhibition of LRRK2 may be a therapeutically beneficial approach for many forms of PD, including idiopathic PD. LRRK2 disease-causing mutations increase kinase activity.

The level of LRRK2-dependent lysosome function can be determined by measuring the abundance of phosphorylated LRRK2 (pS935), phosphorylated ras-related protein Rab 10 (pRab10), or bis(monoacylglycero)phosphate (BMP) (e.g., in the sample, cell, tissue, and/or subject).

### BMP

BMP is a glycerophospholipid that is negatively charged (e.g. at the pH normally present within lysosomes) having the following formula:

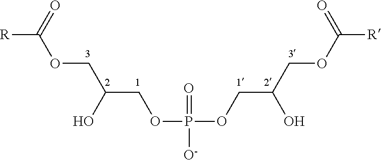

BMP molecules comprise two fatty acid side chains. R and R′ in the above formula represent independently selected saturated or unsaturated aliphatic chains, each of which typically contains 14, 16, 18, 20, or 22 carbon atoms. When a fatty acid side chain is unsaturated, it can contain 1, 2, 3, 4, 5, 6, or more carbon-carbon double bonds. Furthermore, a BMP molecule can contain one or two alkyl ether substituents, wherein the carbonyl oxygen of one or both fatty acid side chains is replaced with two hydrogen atoms.

Nomenclature used herein to describe a particular BMP species refers to a species having two fatty acid side-chains, wherein the structures of the fatty acid side chains are indicated within parentheses in the BMP format (e.g., BMP(18:1_18:1)). The numerals follow the standard fatty acid notation format of number of “fatty acid carbon atoms: number of double bonds.” An “e-” prefix is used to indicate the presence of an alkyl ether substituent wherein the carbonyl oxygen of the fatty acid side chain is replaced with two hydrogen atoms. For example, the “e” in “BMP(16:0e_18:0)” denotes that the side chain having 16 carbon atoms is an alkyl ether substituent.

BMP is unusual in that it has an sn-1:sn-1′ structural configuration (i.e., based on the phosphate-linked glycerol carbon) that is not observed in other glycerophospholipids. Synthesis of BMP involves a number of acylation and diacylation steps and involves transacylase activity, which reorients the glycerol backbone and produces the unusual structural configuration. The sn-1;sn-1′ configuration is believed to contribute to the resistance of BMP to cleavage by many phospholipases and its stability in late endosomes and lysosomes. While BMP is found in many different cell types in low amounts. BMP content is significantly higher in macrophages, as well as lysosomes in liver and other tissue types.

Consistent with their function as digestive organelles, lysosomes contain large amounts of hydrolytic enzymes at an acidic pH (i.e., a pH of about 4.6 to about 5). Various cellular constituents and foreign antigens are captured by receptors on the cell surface for uptake and delivery to lysosomes. Within the cell, receptors such as the mannose-6-phosphate receptor bind and divert hydrolytic enzymes from biosynthetic pathways to the lysosomes. The captured molecules pass through an intermediate heterogeneous set of organelles known as endosomes, which function as a sorting station where the receptors are recycled before hydrolases and other materials are directed to the lysosomes. There, the hydrolases are activated and the unwanted materials are digested. In particular, internal membranes of mature or “late” endosomes and lysosomes contain large amounts of BMP.

Being negatively-charged at lysosomal pH, BMP can dock with luminal acid hydrolases that are positively charged at acidic pH and require a water-lipid interface for activation. By binding in this way, BMP can stimulate a number of lysosomal lipid-degrading enzymes, including acid sphingomyelinase, acid ceramidase, acid phospholipase A2, and an acid lipase that has the capacity to hydrolyze triacylglycerols and cholesterol esters.

Endosomal membranes are a continuation of lysosomal membranes, and they function to sort and recycle material back to the plasma membrane and endoplasmic reticulum. Accordingly, low-density lipoproteins (LDLs) that are internalized in the liver reach late endosomes, where the constituent cholesterol esters are hydrolyzed by an acidic cholesterol ester hydrolase. The characteristic network of BMP-rich membranes contained within late endosomes is an important element of cholesterol homeostasis in that it regulates cholesterol transport by acting as a collection and re-distribution point for free cholesterol. For example, when lysosomal membranes are incubated with anti-BMP antibodies, substantial amounts of cholesterol accumulate.

In some embodiments of methods of the present disclosure, the abundance of a single BMP species are measured. In some embodiments, the abundance of two or more BMP species is measured. In some embodiments, the abundance of at least two, three, four, five, or more of the BMP species are measured. When the abundance of two or more BMP species is measured, any combination of different BMP species can be used.

In some cases, one or more BMP species may be differentially expressed (e.g. more or less abundant) in one type of sample when compared to another, such as, for example, cell-based samples (e.g., cultured cells) versus tissue-based or blood samples. Accordingly, in some embodiments, the selection of the one or more BMP species (i.e., for the measurement of abundance) depends on the type of sample. In some embodiments, the one or more BMP species comprise BMP(18:1_18:1), e.g., when a sample (e.g., a test sample and/or a reference sample) is bone marrow-derived macrophage (BMDM). In other embodiments, the one or more BMP species comprise BMP(22:6_22:6), e.g., when a sample comprises tissue (e.g. brain tissue, liver tissue) or plasma, urine, or CSF.

In some embodiments, an internal BMP standard (e.g., BMP(14:0_14:0)) is used to measure the abundance of one or more BMP species in a sample and/or determine a reference value (e.g., measure the abundance of one or more BMP species in a reference sample). For example, a known amount of the internal BMP standard can be added to a sample (e.g., a test sample and/or a reference sample) to serve as a calibration point such that the amount of one or more BMP species that are present in the sample can be determined. In some embodiments, a reagent used in the extraction or isolation of BMP from a sample (e.g., methanol) is “spiked” with the internal BMP standard. Typically, the internal BMP standard will be one that does not naturally occur in the subject.

Typically, the abundance of each of the one or more BMP species in a test sample will be compared to one or more reference values (e.g., a corresponding reference value). In some embodiments, a BMP value is measured before treatment and at one or more time points after treatment. The abundance value taken at a later time point can be compared to the value prior to treatment as well as to a control value, such as that of a healthy or diseased control, to determine how the subject is responding to the therapy. The one or more reference values can be from different cells, tissues, or fluids corresponding to the cell, tissue, or fluid of the test sample.

In some embodiments, the reference value is the abundance of the one or more BMP species that is measured in a reference sample. The reference value can be a measured abundance value (e.g., abundance value measured in the reference sample), or can be derived or extrapolated from a measured abundance value. In some embodiments, the reference value is a range of values, e.g., when the reference values are obtained from a plurality of samples or a population of subjects. Furthermore, the reference value can be presented as a single value (e.g., a measured abundance value, a mean value, or a median value) or a range of values, with or without a standard deviation or standard of error.

In some embodiments, both the first test sample and the second test sample are obtained from a subject (e.g., a target subject) after the subject has been treated, i.e., the first test sample is obtained from the subject at an earlier time point during treatment than the second test sample. In some embodiments, the first test sample is obtained before the subject has been treated for Parkinson's disease with a LRRK2 inhibitor and the second test sample is obtained after the subject has been treated for the disorder with a LRRK2 inhibitor (i.e., a post-treatment test sample). In some embodiments, more than one (e.g., 1, 2, 3, 4, 5, 6, 7, 8, 9, 10, or more) pre-treatment and/or post-treatment test samples are obtained from the subject. Furthermore, the number of pre-treatment and post-treatment test samples that are obtained need not be the same.

Di-docosahexaenoyl (22:6) bis(monoacylglycerol)phosphate (di-22:6-BMP) is a LRRK2-dependent indicator of lysosome function and dysfunction (Fuji et al. 2015; Liu, N. et al, (2014) Toxicol. Appl. Pharmacol. 279:467-476; U.S. Pat. No. 8,313,949), having the structure:

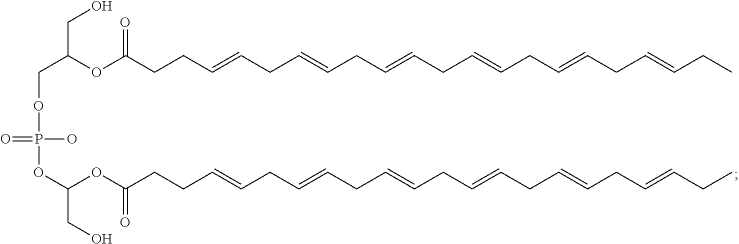

and named as: 1-(((1-(((4E,7E,10E,13E,16E,19E)-docosa-4,7,10,13,16,19-hexaenoyl)oxy)-2-hydroxyethoxy)(11-oxidaneyl)phosphoryl)oxy)-3-hydroxypropan-2-yl (4E,7E,10E,13E,16E,19E)-docosa-4,7,10,13,16,19-hexaenoate. The class of glycerophosphate lipids are susceptible to rapid acyl migration, resulting in phosphate ester exchange and racemization of stereocenters.
pRab10

Mutations in the gene encoding leucine-rich repeat kinase 2 (LRRK2) are found in both familial and non-familial (sporadic) forms of Parkinson's disease (PD). Several different mutations have been identified as pathogenic mutations, including the mutations 11122V, N1437H, R1441C/G/H, RI 728H, R1628P, Y1699C, G2019S, 12020T, T2031S, and G2385R, and other mutations in LRRK2 are associated with susceptibility to PD. At least some of the known pathogenic mutations in LRRK2 have been found to affect its kinase activity, and accordingly, LRRK2 inhibitors have been proposed as a treatment for PD.

Several proteins have been identified as possible physiological substrates of LRRK2, including Rab10, which is a member of the Rab GTPase family. Phosphorylation of the Rab protein is detected in human cells that overexpress LRRK2 and Rab10. Furthermore, increased phosphorylation of Rab10 is detected m different PD-linked LRRK2 mutants, relative to wild-type LRRK2. The enhanced phosphorylation of Rab10 in the presence of LRRK2 variants suggests that there is increased LRRK2 kinase activity in pathogenic variants in vivo. Thus, in some embodiments, phosphorylation of Rab10 represents a useful clinical marker for identifying patients having a pathogenic mutation in LRRK2, such as a 11122V, N1437H, R1441C/G/H, RI 728H, R1628P, Y1699C, G2019S, I2020T, T2031S or G2385R mutation, and in another embodiment, a R1441C, R1441G. Y1699C, G2019S, or I2020T mutation.

Monoclonal antibodies have been generated that specifically bind to phosphorylated Rab10 protein that is endogenously expressed in a human biological sample, such as human peripheral blood mononuclear cells. See PCT/US2018/037809, filed on Jun. 15, 2018 and published as WO 2018/232278 on Dec. 20, 2018, which is hereby incorporated by reference in its entirety for all purposes. In contrast, known polyclonal antibodies against phosphorylated Rab10 or phosphorylated Rab8a do not exhibit a significant decrease in detectable phosphorylated Rab10, in response to treatment with a LRRK2 inhibitor. It has also been found that the levels of phosphorylated Rab10 and phosphorylated Rab8a protein decrease in a dose-dependent manner in response to treatment with a LRRK2 inhibitor, as measured using an anti-phosphorylated Rab10 monoclonal antibody.

pS935

The G2019S mutation noted above is in the activation loop of LRRK2 and is the most common genetic cause of PD. G2019S causes an increase in LRRK2 kinase activity, resulting in toxicity. A marker for LRRK2 activity is phosphorylation of serine 935 (pS935). pS935 is reduced in response to all known LRRK2 kinase inhibitors and thus is a useful biomarker therefor.

BMP Detection Techniques: In some embodiments, mass spectrometry (MS) is used to detect and/or measure the abundance of one or more BMP species according to methods of the present disclosure. Mass spectrometry is an established technique in which compounds are ionized, and the resulting ions are sorted by their mass-to-charge ratios (abbreviated m/Q, m/q, m/Z, or m/z). A sample (e.g., comprising a BMP molecule), which can be present in gas, liquid, or solid form, is ionized, and the resulting ions are then accelerated through an electric and/or magnetic field, causing them to be separated by their mass-to-charge ratios. The ions ultimately strike an ion detector and a mass spectrogram is generated. The mass-to-charge ratios of the detected ions, together with their relative abundance, can be used to identify the parent compound(s), sometimes by correlating known masses (e.g., of entire or intact molecules) to the masses of the detected ions and/or by recognition of patterns that are detected in the mass spectrogram.

In some embodiments, high performance liquid chromatography (HPLC), is used in combination with mass spectrometry. HPLC provide a high degree of separation by forcing the analyte in a mobile phase under pressure through a stationary phase, typically a densely packed column. HPLC functions as the separation front end and mass spectrometry as the characterization back end in the established technique of LC/MS.

pRab10 and pS935 Detection

As discussed for BMP above, pRab10 and pS935 can also be detected using MS. However, in one embodiment of the invention, as described in the Examples below, pRab10 and pS935 are detected using antibodies specific for those molecules. Those antibodies can be used for detection in immunoassays. One such commercial assay is sold by Meso Scale Diagnostics. LLC. (MSD) in Rockville, Maryland.

### Method for Treating Parkinson's Disease

Methods for treating diseases or conditions mediated, at least in part, by LRRK2, are described generally in U.S. Pat. No. 10,590,114, and compounds for use in such methods are described in U.S. Pat. No. 9,932,325, both of which are incorporated by reference herein in their entireties for all purposes.

A method is provided for treating Parkinson's disease, the method comprising administering to a subject in need thereof between about 70 to 800 mg/day of a LRRK2 inhibitor that is

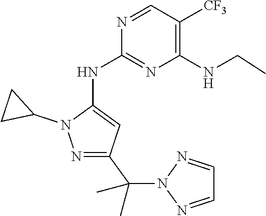 [SMILES: CCNc1nc(Nc2cc(C(C)(C)n3nccn3)nn2C2CC2)ncc1C(F)(F)F]

or a pharmaceutically acceptable salt or deuterated analog thereof.

The daily dosage may be described as a total amount of compound I or a pharmaceutically acceptable salt or deuterated analog thereof administered per dose or per day. Daily dosage of compound I or a pharmaceutically acceptable salt or deuterated analog thereof may be between about 70 to 800 mg, between about 70 to 225 mg/day, or between about 70 and 80 mg/day.

In particular embodiments, the dose may be 70, 75, 80, 105, 130, 150, 225, 250, 300 or 400 mg. In some embodiments, the compound or a pharmaceutically acceptable salt or deuterated analog thereof may be administered once daily (QD). In other embodiments the administration is twice daily (BID).

In some embodiments, provided is a pharmaceutical composition comprising about 75 mg of compound I in a tablet form.

In some embodiments, two tablets each comprising about 75 mg of compound I are administered to a subject in need thereof. In some embodiments, two tablets each comprising about 75 mg of compound I are administered once per day to a subject in need thereof for a total dose of about 150 mg/day.

In some embodiments, three tablets each comprising about 75 mg of compound I are administered to a subject in need thereof. In some embodiments, three tablets each comprising about 75 mg of compound I are administered once per day to a subject in need thereof for a total dose of about 225 mg/day.

In other embodiments, the compounds of the present disclosure can be administered in combination with an additional agent having activity for treatment of Parkinson's disease. For example, in some embodiments the compounds are administered in combination with one or more additional therapeutic agents useful for treatment of Parkinson's disease. In some embodiments, the additional therapeutic agent is L-dopa (e.g., Sinemet®), a dopaminergic agonist (e.g. Ropinerol or Pramipexole), a catechol-O-methyltransferase (COMT) inhibitor (e.g. Entacapone), a L-monoamine oxidase (MAO) inhibitor (e.g., selegiline or rasagiline) or an agent which increases dopamine release (e.g., Zonisamide).

Method for Treating Parkinson's Disease with a LRRK2 Inhibitor

In one embodiment, a method is provided for treating Parkinson's disease, the method comprising administering once a day to a subject in need thereof between about 75 to 225 mg of compound I:

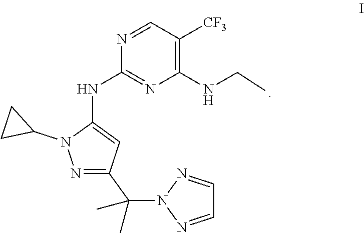 [SMILES: CCNc1nc(Nc2cc(C(C)(C)n3nccn3)nn2C2CC2)ncc1C(F)(F)F]

In another embodiment, provided is a method for treating Parkinson's disease, the method comprising administering once a day to a subject in need thereof a pharmaceutical composition comprising between about 75 to 225 mg of compound I:

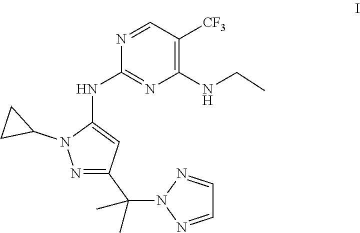 [SMILES: CCNc1nc(Nc2cc(C(C)(C)n3nccn3)nn2C2CC2)ncc1C(F)(F)F]

or a pharmaceutically acceptable salt or deuterated analog thereof and a pharmaceutically acceptable carrier.
Method for Reducing Phosphorylated S935 LRRK2 (PS935) in Whole Blood of a Subject Suffering from Parkinson's Disease

In one embodiment, a method is provided for reducing phosphorylated S935 LRRK2 (pS935) in whole blood of a subject suffering from Parkinson's disease, the method comprising administering to a subject in need thereof between about 70 to 800 mg/day of compound I:

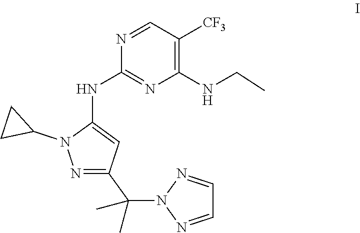 [SMILES: CCNc1nc(Nc2cc(C(C)(C)n3nccn3)nn2C2CC2)ncc1C(F)(F)F]

or a pharmaceutically acceptable salt or deuterated analog thereof.

In another embodiment, a method is provided for reducing phosphorylated S935 LRRK2 (pS935) in whole blood of a subject suffering from Parkinson's disease, the method comprising administering to a subject in need thereof a pharmaceutical composition comprising between about 70 to 800 mg/day of compound I:

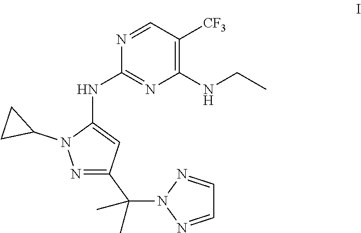 [SMILES: CCNc1nc(Nc2cc(C(C)(C)n3nccn3)nn2C2CC2)ncc1C(F)(F)F]

or a pharmaceutically acceptable salt or deuterated analog thereof and a pharmaceutically acceptable carrier.
Method for Reducing Phosphorylated Ras-Related Protein RAB10 (PRAB10) in Peripheral Blood Mononuclear Cells (PBMC) of a Subject Suffering from Parkinson's Disease

In one embodiment, a method is provided for reducing phosphorylated ras-related protein Rab10 (pRab10) in peripheral blood mononuclear cells (PBMC) of a subject suffering from Parkinson's disease, the method comprising administering to a subject in need thereof between about 70 to 800 mg/day of compound I:

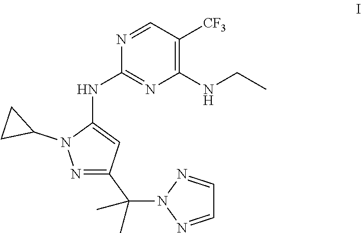 [SMILES: CCNc1nc(Nc2cc(C(C)(C)n3nccn3)nn2C2CC2)ncc1C(F)(F)F]

or a pharmaceutically acceptable salt or deuterated analog thereof.

In another embodiment, a method is provided for reducing phosphorylated ras-related protein Rab10 (pRab10) in peripheral blood mononuclear cells (PBMC) of a subject suffering from Parkinson's disease, the method comprising administering to a subject in need thereof a pharmaceutical composition comprising between about 70 to 800 mg/day of compound I:

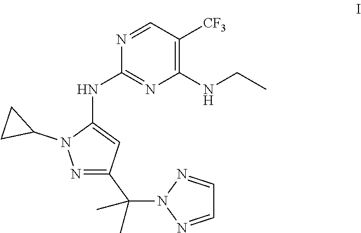 [SMILES: CCNc1nc(Nc2cc(C(C)(C)n3nccn3)nn2C2CC2)ncc1C(F)(F)F]

or a pharmaceutically acceptable salt or deuterated analog thereof and a pharmaceutically acceptable carrier.
Method for Reducing Lysosomal Lipid 22:6-Bis[monoacylglycerol]phosphate (BMP) in Urine of a Subject Suffering from Parkinson's Disease

In one embodiment a method is provided for reducing lysosomal lipid 22:6-bis[monoacylglycerol]phosphate (BMP) in urine of a subject suffering from Parkinson's disease, the method comprising administering to a subject in need thereof between about 70 to 800 mg/day of compound I:

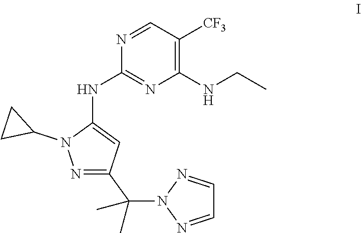 [SMILES: CCNc1nc(Nc2cc(C(C)(C)n3nccn3)nn2C2CC2)ncc1C(F)(F)F]

or a pharmaceutically acceptable salt or deuterated analog thereof.

In another embodiment, a method is provided for reducing lysosomal lipid 22:6-bis[monoacylglycerol]phosphate (BMP) in urine of a subject suffering from Parkinson's disease, the method comprising administering to a subject in need thereof a pharmaceutical composition comprising between about 70 to 800 mg/day of compound I:

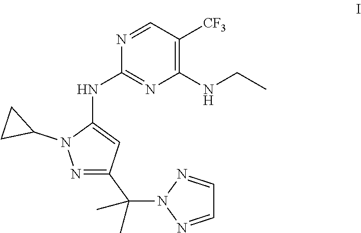 [SMILES: CCNc1nc(Nc2cc(C(C)(C)n3nccn3)nn2C2CC2)ncc1C(F)(F)F]

or a pharmaceutically acceptable salt or deuterated analog thereof and a pharmaceutically acceptable carrier.

### Use of a LRRK2 Inhibitor for Treating Parkinson's Disease

A use is provided of a LRRK2 inhibitor for treating Parkinson's disease, wherein the inhibitor is administered to a subject in need thereof between about 70 to 800 mg/day and is

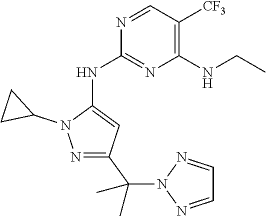 [SMILES: CCNc1nc(Nc2cc(C(C)(C)n3nccn3)nn2C2CC2)ncc1C(F)(F)F]

or a pharmaceutically acceptable salt or deuterated analog thereof.

### Use of a LRRK2 Inhibitor in the Manufacture of a Medicament for Treating Parkinson's Disease

A use is provided of a LRRK2 inhibitor in the manufacture of a medicament for treating Parkinson's disease, wherein the inhibitor is administered to a subject in need thereof between about 70 to 800 mg/day and is

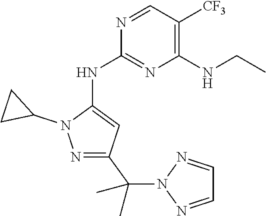 [SMILES: CCNc1nc(Nc2cc(C(C)(C)n3nccn3)nn2C2CC2)ncc1C(F)(F)F]

or a pharmaceutically acceptable salt or deuterated analog thereof.

### Method for Monitoring a Response to a LRRK2 Inhibitor Compound

A method is provided for monitoring a subject's response to the treatment methods provided herein, the method comprising:
(a) measuring an amount of one or more pS935, pRab10 or BMP species in a test sample from a subject having Parkinson's disease, wherein the test sample or subject has been treated with between about 70 to 800 mg/day of compound I or a pharmaceutically acceptable salt or deuterated analog thereof;
(b) comparing the difference in amount between the one or more BMP species measured in (a) and one or more reference values, and
(c) determining from the comparison whether the LRRK2 inhibitor compound or pharmaceutical composition thereof, or dosing regimen thereof improves one or more BMP species levels for treating the Parkinson's disease.

In one embodiment, the method further comprises
(d) maintaining or adjusting the amount or frequency of administration of compound I or a pharmaceutically acceptable salt or deuterated analog thereof to the test sample or subject; and
(e) administering the compound or a pharmaceutically acceptable salt or deuterated analog thereof to the test sample or to the subject.

In an exemplary embodiment, the one or more BMP species comprise BMP(22:6_22:6).

In an exemplary embodiment, the LRRK2 inhibitor is compound I or a pharmaceutically acceptable salt or deuterated analog thereof.

In an exemplary embodiment, the one or more BMP species comprise BMP(22:6_22:6).

In exemplary embodiments of the methods described above, the reference value is measured in a reference sample obtained from a reference subject or a population of reference subjects.

In exemplary embodiments of the methods described above, the reference subject or population of reference subjects is a healthy control.

In exemplary embodiments of the methods described above, the reference subject or population of reference subjects do not have a lysosomal dysfunction disorder or a decreased level of pS935, pRab10 or BMP.

In exemplary embodiments of the methods described above, a subject having, or at risk of having. Parkinson's disease has increased pS935, pRab10 or BMP species levels in bone marrow derived macrophages compared to a healthy control or a control not related to a Parkinson's disease.

In exemplary embodiments of the methods described above, a subject having, or at risk of having, Parkinson's disease has decreased pS935, pRab10 or BMP species levels in liver, brain, cerebrospinal fluid, plasma, or urine compared to a healthy control or a control not related to a Parkinson's disease.

In exemplary embodiments of the methods described above, the amount of a pS935, pRab10 or BMP species in the test sample of a subject having, or at risk of having, a Parkinson's disease has at least about a 1.2-fold, 1.5-fold, or 2-fold difference compared to a reference value of a control such as a healthy control or a control not related to Parkinson's disease.

In exemplary embodiments of the methods described above, the amount of a pS935, pRab10 or BMP species in the test sample of a subject having, or at risk of having, a Parkinson's disease is about a 1.2-fold to about 4-fold difference compared to a reference value of a control such as a healthy control or a control not related to a lysosomal dysfunction disorder.

In exemplary embodiments of the methods described above, the reference value is the pS935, pRab10 or BMP species value prior to treatment.

In exemplary embodiments of the methods described above, the reduced pS935, pRab10 or BMP species level is an improvement over the pS935, pRab10 or BMP species level prior to treatment relative to the reference value of a control such as a healthy control or a control not related to a lysosomal dysfunction disorder.

In exemplary embodiments of the methods described above, the reduced pS935, pRab10 or BMP species level has a difference compared to the control of 5% to 90%, preferably about 50-70%, greater than about 50%, or greater than about 70%.

In exemplary embodiments of the methods described above, the test or reference sample or one or more reference values comprise or relate to a cell, a tissue, whole blood, plasma, serum, cerebrospinal fluid, interstitial fluid, sputum, urine, lymph, or a combination thereof.

In exemplary embodiments of the methods described above, the cell is a peripheral blood mononuclear cell (PBMC), a bone marrow-derived macrophage (BMDM), a retinal pigmented epithelial (RPE) cell, a blood cell, an erythrocyte, a leukocyte, a neural cell, a microglial cell, a brain cell, a cerebral cortex cell, a spinal cord cell, a bone marrow cell, a liver cell, a kidney cell, a splenic cell, a lung cell, an eye cell, a chorionic villus cell, a muscle cell, a skin cell, a fibroblast, a heart cell, a lymph node cell, or a combination thereof.

In exemplary embodiments of the methods described above, the cell is a cultured cell.

In exemplary embodiments of the methods described above, the tissue comprises brain tissue, cerebral cortex tissue, spinal cord tissue, liver tissue, kidney tissue, muscle tissue, heart tissue, eye tissue, retinal tissue, a lymph node, bone marrow, skin tissue, blood vessel tissue, lung tissue, spleen tissue, valvular tissue, or a combination thereof.

In exemplary embodiments of the methods described above, the test sample comprises an endosome, a lysosome, an extracellular vesicle, an exosome, a microvesicle, or a combination thereof.

In exemplary embodiments of the methods described above, the one or more pS935, pRab10 or BMP species comprise two or more pS935, pRab10 or BMP species.

In exemplary embodiments of the methods described above, the test sample comprises plasma, urine, cerebrospinal fluid (CSF), and/or brain or liver tissue, and the one or more BMP species comprise BMP(22:6_22:6).

In exemplary embodiments of the methods described above, the test sample comprises CSF or urine and the one or more BMP species comprise BMP(22:6_22:6).

In exemplary embodiments of the methods described above, the abundance of the one or more pS935, pRab10 or BMP species is measured using liquid chromatography-mass spectrometry (LC-MS), liquid chromatography-tandem mass spectrometry (LC-MS/MS), gas chromatography-mass spectrometry (GC-MS), gas chromatography-tandem mass spectrometry (GC-MS/MS), enzyme-linked immunosorbent assay (ELISA), or a combination thereof.

In exemplary embodiments of the methods described above, an internal pS935, pRab10 or BMP standard is used when measuring the amount of the one or more pS935, pRab10 or BMP species.

In exemplary embodiments of the methods described above, the internal pS935, pRab10 or BMP standard comprises a pS935, pRab10 or BMP species that is not naturally present in the subject and/or the reference subject or population of reference subjects.

In exemplary embodiments of the methods described above, the internal BMP standard comprises BMP(14:0_14:0).

In exemplary embodiments of the methods described above, the lysosomal dysfunction disorder is a disorder related to BMP expression, processing, glycosylation, cellular uptake, trafficking, and/or function.

In exemplary embodiments of the methods described above, the subject has one or more mutations in a LRRK2-expressing gene.

In exemplary embodiments of the methods described above, the disorder is associated with decreased BMP level in tissues.

In exemplary embodiments of the methods described above, the disorder is associated with increased BMP level in urine.

In exemplary embodiments of the methods described above, the disorder is associated with increased BMP level in urine.

In exemplary embodiments of the methods described above, the subject and/or the reference subject is a human, a non-human primate, a rodent, a dog, or a pig.

### Pharmaceutical Compositions and Modes of Administration

Provided herein are pharmaceutical compositions that contain compound I or a pharmaceutically acceptable salt or deuterated analog thereof and one or more pharmaceutically acceptable vehicles selected from carriers, adjuvants, and excipients. Suitable pharmaceutically acceptable vehicles may include, for example, inert solid diluents and fillers, diluents, including sterile aqueous solution and various organic solvents, permeation enhancers, solubilizers, and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker. Inc. 3rd Ed. (G. S. Banker & C. T. Rhodes. Eds.).

Oral administration may be via, for example, capsule or tablets. In making the pharmaceutical compositions the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Some examples of suitable excipients include, e.g., lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

### EXAMPLES

The compositions and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the processes, formulations and/or methods of the invention may be made without departing from the spirit of the invention and the scope of the appended claims.

### Disease Overview

PD is the second most common neurodegenerative disease, affecting approximately 1% to 2% of individuals aged 65 years or over (de Rijk M D, et al., J Neurol Neurosurg Psychiatry. 1997; 62(1):10-5; Blin P et al., Eur J Neurol. 2015; 22(3):464-71), and the prevalence is projected to increase substantially as the global populations age (Dorsey E R et al, Neurology. 2007; 68(5):384-6). The estimated prevalence of PD in Europe and North America ranges from 66 to 12,500 per 100,000 (von Campenhausen et al., Eur Neuropsychopharmacol. 2005; 15(4):473-90) and 572 per 100,000 (Marras et al., NPJ Parkinsons Dis. 2018; 4:21), respectively. The incidence of PD increases with age, being rare before age 50 years (de Lau and Breteler, Lancet Neurol. 2006; 5(6):525-35; Twelves et al., Mov Disord. 2003:18(1):19-31). Severe disability or death may be expected in 35% of patients within 5 years of onset, 65% of patients within 10 years of onset, and 80% of patients within 15 years of onset (Poewe, J Neurol. 2006:253 Suppl 7:VII2-6; Schrag and Bank, Mov Disord. 2006; 21(11):1839-43; Mov Disord. 2010:25 Suppl 1:S131-5).

Currently approved treatments for PD improve motor symptoms but do not address the underlying cause of the disease. Over time, these symptomatic therapies lose effectiveness and are associated with increasing frequency and severity of adverse effects, such as dyskinesias and hallucinations. In addition, nonmotor symptoms, including depression, anxiety, sleep disorders, cognitive impairment, and dementia, are disabling and common features of PD, but are poorly addressed by current therapies (Aarsland et al., Arch Neurol. 1996:53(6):538-42; Truong et al., J Neural Sci. 2008:266(1-2):216-28; Lyons and Pahwa, Am J Manag Care. 2011; 17 Suppl 12:5308-14; Khoo et al., Neurology. 2013; 80(3):276-81; Seppi et al., Mov Disord. 2019; 34(2):180-98; FDA 2016). As such, PD patients inevitably experience mounting disabilities over the years to decades that they live with the disease (Helv et al., Mov Disord. 2005:20(2):190-9). Thus, there is a significant need for an effective disease-modifying therapy to prevent the progressive motor and nonmotor disabilities not addressed by current therapies.

LRRK2 mutations are an established cause of PD, accounting for approximately 4% to 5% of familial PD (Healy et al., Lancet Neurol. 2008; 7(7):583-90; Chai C, et al., Curr Genomics 2013; 14(8):486-501). Familial LRRK2 mutations are transmitted in an autosomal dominant pattern of inheritance with incomplete penetrance (Marder et al., Neurology 2015; 85(1):89-95). In addition, variants within the LRRK2 gene are a genetic risk factor and account for 1% to 2% of “sporadic” PD cases (Healy, 2008; Chai et al, Curr Genomics 2013; 14(8):486-501; Hernandez et al., J Neurochem 2016; 139(Suppl 1):50-74: Cookson, Biochem Soc Trans. 2016; 44(6):1603-10).

Rationale for Treatment with Investigational Agent in Disease

Compound 1 is a selective, orally bioavailable, CNS-penetrant, reversible inhibitor of LRRK2 for treatment of patients with PD. Inhibition of LRRK2 kinase, a genetically validated target, improves lysosomal function in LRRK2-PD, as well as potentially in iPD. Compound 1 may intervene in an important disease pathway in PD and prevent or curb the accumulation of motor and non-motor disabilities that define the progression of PD.

LRRK2 encodes a multidomain protein containing a guanosine triphosphatase (GTPase) domain, a kinase domain, and several potential protein-protein interaction domains. The majority of identified pathogenic mutations in LRRK2 are located within its catalytic domains, including the most common mutation associated with LRRK2-PD, G2019S. These mutations increase LRRK2 kinase activity, either through direct mechanisms within the kinase domain, or through indirect mechanisms (West et al., Hunan Mol Gen. 2007; 16(2):223-32: Sheng et al., Sci Trans/Med. 2012; 4(164):164ra161). The G2019S point mutation increases LRRK2 activity by approximately 2-fold, and a protective LRRK2 variant is associated with a subtle reduction in LRRK2 kinase activity, suggesting that modest changes in LRRK2 kinase activity contribute to the life-time risk of PD (Khan et al., Brain. 2005; 128(Pt 12):2786-96: Jaleel et al., Biochem J. 2007; 405(2):307-17; West et al., Human Mol Gen. 2007:16(2):223-32: Sheng et al., Sci Trans/Med. 2012:4(164):164ra161; Steger et al., eLife. 2016; 5:e12813; Ross et al., Lancet Neurol. 2011; 10(10):898-908).

While the exact pathogenic mechanisms remain unknown, LRRK2 is believed to play a role in intracellular trafficking in the endo-lysosomal system (Henry et al. 2015: Cookson et al., 2015). A direct effect of kinase-activating LRRK2 mutations is to increase phosphorylation of Rab GTPases, important regulators of intracellular trafficking (Steger et al., 2016;). Rab phosphorylation is thought to promote accumulation of inactive Rab in lysosomal membranes and consequently perturb vesicle trafficking. Alterations in both lysosomal function and cellular function are associated with LRRK2 mutations. Cellular data show that inhibition of G2019S mutant LRRK2 activity in cells reverses the lysosomal abnormalities (Khan et al., 2005; West et al., 2007; Sheng et al., 2012: Steger et al., 2016; Schapansky et al., Neurobiol Dis. 2018.111:26-35; Hockey et al., J Cell Sci. 2015; 128(2):232-8; Henry et al., 2015; Wallings et al., Hum Mol Genet. 2019a; 28(16):2696-710; Rivero-Rios et al., J Biol Chem. 2019; 294(13):4738-58).

Current evidence supports LRRK2 inhibition for correcting disease-associated lysosome dysfunction independent of LRRK2 mutation status. LRRK2 activity, as measured by pS1292 LRRK2 and Rab10 threonine 73 phosphorylation (pT73 Rab10), is increased in the substantia nigra of brains collected postmortem from patients with iPD, suggesting that LRRK2 overactivity may drive pathogenesis of PD in the non-LRRK2-carrier population (Di Maio et al., Sci Transl Med. 2018:10(451): eaar5429). Lysosomal dysfunction may be a central mechanism for accumulation of intracellular proteins resulting in accumulation of α-synuclein and the formation of Lewy bodies, a cardinal pathological feature of iPD (Dehay et al., Mov Disord 2013; 28(6):725-32: Tofaris, Mov Disord 2012:27(11):1364-9). The role of LRRK2 in α-synuclein accumulation and consequent pathology is suggested by in vitro and in vivo studies. Primary neuronal cultures expressing G2019S-LRRK2 develop α-synuclein inclusions that can be reduced by LRRK2 inhibitor treatment. In vivo, infection of a transgenic G2019S-LRRK2 rat model of PD with a virus overexpressing α-synuclein can induce dopaminergic neuron neurodegeneration, and this degeneration can be attenuated by LRRK2 inhibitor treatment (Daher et al., J Biol Chem. 2015; 290(32):19433-44; Volpicelli-Daley et al. J Neurosci. 2016:36(28):7415-27). This is further supported by data showing significant protection from pathology when LRRK2 protein levels are reduced by 50% in a murine model of PD in which pathogenic α-synuclein is overexpressed (Zhao et al. Mol Ther Nucleic Acids. 2017; 8:508-19). These data strongly support the concept that LRRK2 hyperactivity may have an impact on lysosomal function and contribute to neuronal degeneration in iPD, and that kinase inhibitors have the potential to restore lysosomal function and improve patient outcomes in the context of iPD.

Additionally, LRRK2 inhibition may correct disease-associated lysosome dysfunction associated with other genetic variants linked to PD. LRRK2 kinase inhibition can correct signaling deficits, including increased phosphorylated Rab10, associated with PD-linked mutations in the lysosomal trafficking molecules VPS35 and Rab29 (Purlyte et al., EMBO J. 2018; 37(1):1-18; Mir et al., Biochem J. 2018; 475(11):1861-83). Furthermore, patients carrying homozygous loss-of-function mutations in the gene encoding glucosylceramidase β (GBA) develop the lysosomal storage disorder Gaucher disease while subjects harboring heterozygous mutations in GBA have increased risk of PD. In fibroblasts derived from Gaucher disease patients, there is a nearly complete loss of lysosome protein turnover activity, which can be partially corrected by LRRK2 inhibition. Hence, inhibiting increased LRRK2 kinase activity may mitigate LRRK2-mediated pathogenesis, including lysosomal dysfunction, as well as lysosomal dysfunction independent of LRRK2 overactivity, supporting the therapeutic potential of compound I in a broad population of patients with PD (Di Maio et al., 2018, Ysselstein et al., Nat Commun. 2019; 10(1):55702019. Sanyal et al., Front Neurosci. 2020; 14:442).

In summary, LRRK2 activity is linked to central mechanisms of PD (iPD and LRRK2-PD) pathology through its role in lysosomal function, and LRRK2 kinase inhibitors such as compound I represent a new class of therapeutics with the potential to address the underlying biology of PD in patients with and without LRRK2 mutations.

### Example 1: LRRK2 Kinase Inhibition in Compound I Phase 1 Studies

In the Phase 1 study in healthy volunteers, the demographics of participants was as follows:
184 HVs (145 active, 39 placebo) were treated with single or multiple once daily (QD) or twice daily (BID) doses up to 28 days in the following study Parts:
   Part A (SAD; young HVs; n=48): 100% male and median age of 25 (range, 18-50) years;
   Part B (10-day MAD; young HVs; n=80): 99% male and median age of 26.5 (range, 18-50) years;
   Part C (SAD: elderly HVs; n=8): 50% male and median age of 69 (range, 67-74) years;
   Part D (28-day MAD; young HVs; n=17): 100% male and median age of 29 (range, 18-39) years; and
   E (14-day MAD: young HVs: n=31): 100% male and median age 30 (range 18-50) years.

### Example 2: Kinase Activity in Parkinson's Disease and LRRK2 Risk Variants

Current evidence suggests that reduction of LRRK2 kinase activity is a viable therapeutic strategy for the treatment of patients with PD, whether or not associated with familial LRRK2 pathogenic mutations. The majority of identified pathogenic mutations in LRRK2 increase LRRK2 kinase activity, either through direct mechanisms within the kinase domain, or through indirect mechanisms (West et al., 2007; Sheng et al., 2012). LRRK2 kinase activity, as measured by pS1292 LRRK2 and pT73 Rab10, is increased in the substantia nigra of brains collected postmortem from patients with iPD, suggesting that LRRK2 kinase overactivity in the brain may drive pathogenesis of PD in the non-LRRK2 mutation-carrier population (Di Maio et al., 2018). In addition, current evidence suggests that LRRK2 inhibition may correct disease-associated lysosome dysfunction such as reduced GBA activity independent of LRRK2 mutation status (Ysselstein et al., 2019). Thus, LRRK2 kinase inhibitors such as compound I represent a new class of therapeutics with the potential to address the underlying biology of PD in patients with and without LRRK2 mutations.

The most common disease-causing variant of LRRK2 (G2019S) increases LRRK2 kinase activity by approximately 2-fold: therefore, normalization can be expected for LRRK2 kinase activity reduction at 50%.

pS935 LRRK2, a Pharmacodynamic Marker of LRRK2 Kinase Inhibition

pS935 LRRK2 in whole blood is the main pharmacodynamic marker used to quantify LRRK2 inhibition in the compound I clinical studies. pS935 LRRK2 has been demonstrated to be sensitive to pharmacological inhibition of LRRK2 kinase (Fell et al., J Pharmacol Ep Ther. 2015; 355(3):397-409; Fuji et al., 2015; Henderson et al., J Med Chem. 2015; 58(1):419-32), and reduction in pS935 LRRK2 is measurable in whole blood in human subjects after treatment with LRRK2 inhibitors. Furthermore, in animal studies, the exposure-response of pS935 LRRK2 peripherally closely corresponds to that in the CNS (e.g., 50% reduction in pS935 LRRK2 peripherally on average corresponds to approximately 50% reduction centrally), demonstrating that peripheral LRRK2 inhibition is likely to reflect inhibition in the brain in human subjects.

Because the main pathological findings of PD are in the brain, direct quantification of LRRK2 inhibition in the CNS would be preferable to quantification of LRRK2 inhibition peripherally as a pharmacodynamic measure. Investigators in the field are therefore developing assays for quantification of LRRK2 phosphorylation in CSF, a matrix that would reflect LRRK2 inhibition in the brain. As these assays advance, they will be implemented in clinical studies to quantify LRRK2 inhibition in the CNS and measure the relationship between peripheral and central LRRK2 inhibition in human subjects.

Based on the 2-fold increase in kinase activity associated with LRRK2 G2019S point mutations, reduction of whole-blood pS935 LRRK2 of ≥50% at trough in ≥50% of the study subjects represents a minimal pharmacodynamic target. In clinical studies to date, exposures resulting in average reduction in pS935 LRRK2 of 85% to 90% have been demonstrated to be safe and generally well tolerated in healthy subjects and subjects with PD. Specifically, there were no apparent changes in potential LRRK2 on-target renal or pulmonary function for up to 28 days of exposure.

### BMP, a Lysosomal Biomarker Modulated by LRRK2 Activity

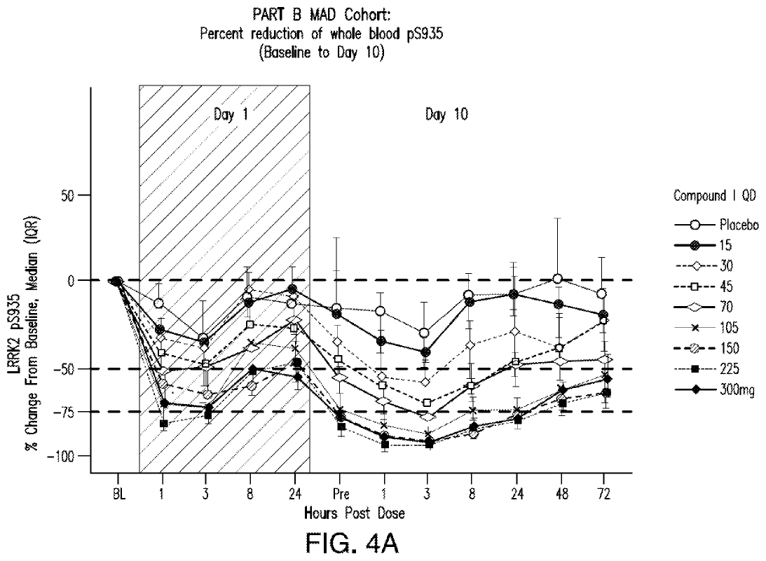

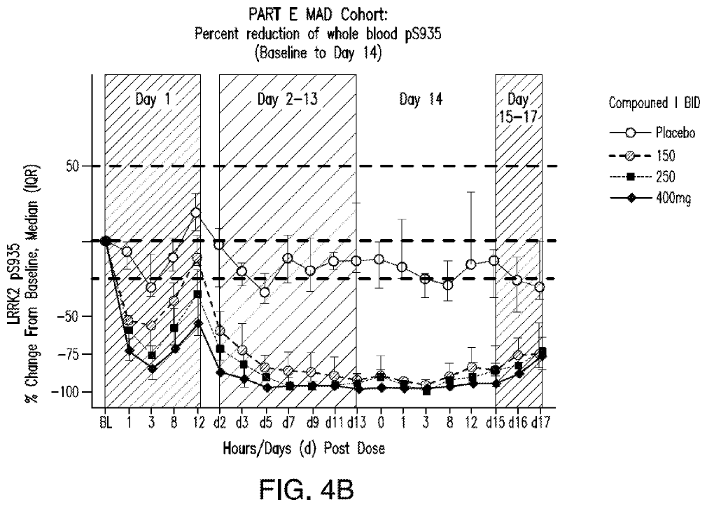

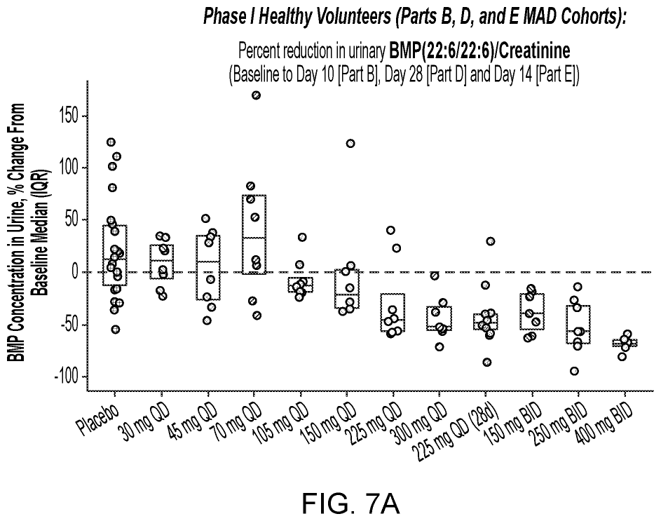

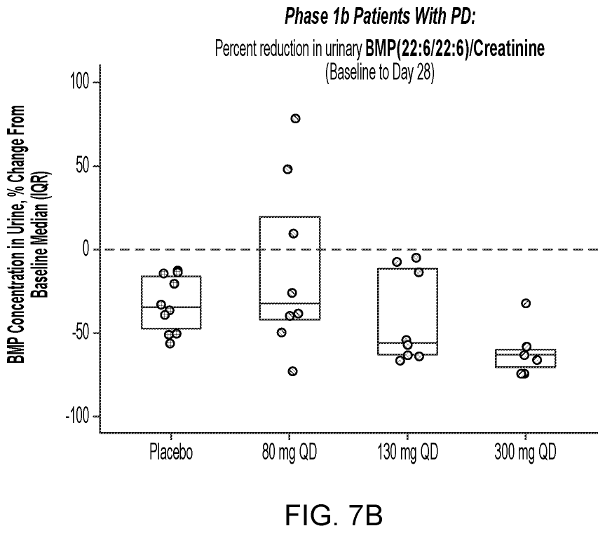

Lysosomal lipid BMP 22:6/22:6 (referred to throughout the document as “BMP”) measured in urine represents a mechanistic marker of correction of the lysosomal pathways downstream of LRRK2. BMP is a lysosomal phospholipid found exclusively on the intraluminal vesicles of late endosomes and lysosomes (Bissig and Gruenberg, Cold Spring Harb Perspect Biol. 2013; 5(10):a016816). Individuals with genetic or drug-induced lysosomal function disorders, including those with G2019S LRRK2 mutations, have increased levels of urine BMP (Lecommandeur et al., J Lipid Res. 2017:58(7): 1306-14; Alcalay et al., Mov Disord. 2013; 28(14):1966-71). LRRK2 inhibition has been shown to reduce urine BMP in both animal models and humans (Fuji et al. 2015: Alcalay et al., 2020; FIGS. 4A, 6A, 7A, 7B).

Lysosomal dysfunction is a common hallmark of PD, and it is hypothesized that therapeutic approaches that aim to improve PD-linked defects in lysosomal homeostasis, including LRRK2 inhibition, may meaningfully affect disease progression (Wallings et al., Trends Neurosci. 2019b; 42(12):899-912). Reductions in urine BMP, though not necessarily required, can indicate modulation of lysosomal pathways that are defective in patients with PD. The main pathological findings in PD are in the CNS; therefore, efficacy of a therapeutic likely requires modulation of lysosomal pathways in the CNS. While there is evidence that urine BMP is a measure of lysosomal function, this is a peripheral marker and therefore additional biomarkers of LRRK2 pathway activity and lysosomal function in the CNS will be investigated to further the connection between urine BMP and central lysosomal function.

### Example 3: Clinical Safety

Safety data from Phase 1 first in human study in healthy subjects and Phase 1b study in subjects with PD are summarized below.

### Phase 1, Safety, Tolerability, Pharmacokinetic, and Pharmacodynamic Study in Healthy Subjects

This was a Phase 1, randomized, placebo-controlled, double-blind FIH study designed to determine the safety, tolerability. PK, and pharmacodynamics of compound I in healthy subjects. Unblinded safety data from multiple-dose cohorts (Parts B, D, and E) are summarized below.

Safety and Tolerability in Healthy Subjects after Multiple-Doses in Part B

Part B of this study was composed of sequential multiple ascending dose (MAD) cohorts. Healthy subjects were enrolled in 8 cohorts (Cohorts B1-B8; n=10/cohort) and were administered compound I 15, 30, 45, 70, 105, 150, 225, or 300 mg or placebo (4:1 ratio) QD for 10 days.

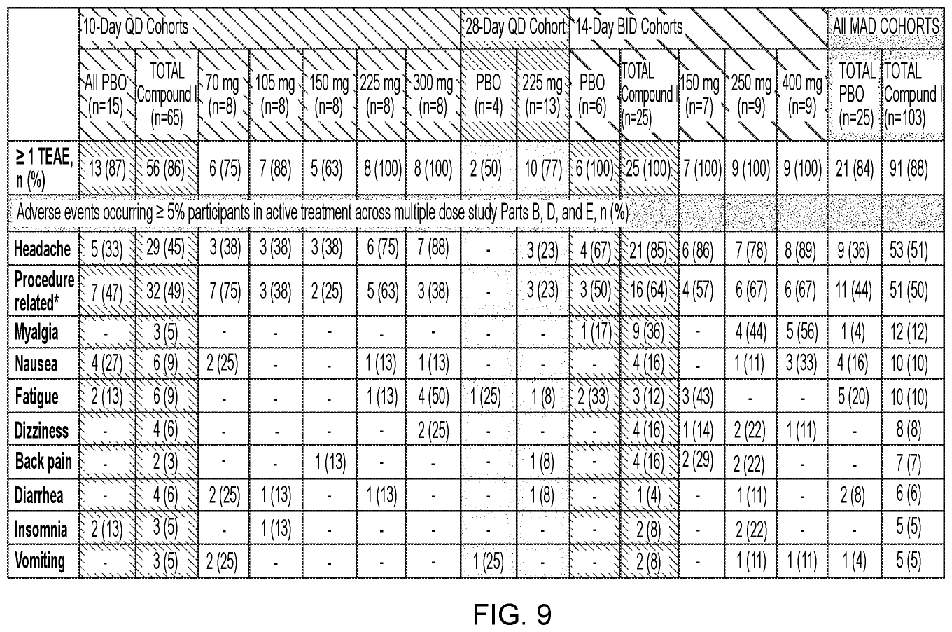

In Part B, Compound I was generally well tolerated at multiple doses up to 300 mg QD for 10 days in healthy subjects. No deaths, other SAEs (serious adverse events), AESIs (adverse events of special interests), or treatment-emergent adverse events (TEAEs) leading to study drug discontinuation were reported. A total of 56 (86.2%) compound I treated subjects and 13 (86.7%) placebo-treated subjects experienced ≥1 TEAE (FIG. 9). The most common TEAEs among compound 1-treated subjects were headache (29 [45%] subjects); procedural related (32 [49%] subjects): fatigue (6 [9.2%] subjects); and nausea (6 [9.2%] subjects). Among the compound I-treated subjects, 55 (84.6%) experienced ≥1 mild TEAE, 9 (13.8%) subjects experienced ≥1 moderate TEAE, and no subjects experienced a severe TEAE.

No clinically significant changes in safety laboratory, vital signs, ECG, neurological examination, pulmonary function test (PFT), or Columbia-Suicide Severity Rating Scale (C-SSRS) results were observed.

Effect of Treatment on pS935 and pRab10

Procedure for pS935 in whole blood and pRab10 in PBMCs:

### Sample Preparation

Whole Blood Preparation for pS935 Assay:

Frozen human whole blood samples were thawed and directly lysed in 96-well plates (100 μl whole blood sample with 100 μl lysis buffer). Samples were spun down (2,500×g for 20 minutes at 4° C.) before MSD assay.

PBMC Preparation for pRab10 Assay:

Blood was collected into CPT-sodium heparin tubes (BD BDAM362780) and PBMCs were then isolated following the manufacturer's protocol. The PBMCs were pelleted by centrifugation at maximum speed and then resuspended in PBMC lysis buffer (1× cell lysis buffer [CST catalog #9803] with PhosSTOP phosphatase inhibitor [Roche 04906837001], complete protease inhibitor [Roche 04693159001], and Benzonase [Sigma E8263]). The lysates were held on ice for 20 minutes, followed by centrifugation at maximum speed at 4° C. for 20 minutes. Supernatants were aliquoted and stored at −80° C. for later immunoassay analysis.

### MSD Assay

Capture antibodies were biotinylated using EZ-Link™ NHS-LC-LC-Biotin (Thermo Fisher, #21343), and detection antibodies were conjugated using Sulfo-TAG NHS-Ester (MSD, R31AA-1). 96-well (or 384-well) MSD GOLD Small Spot Streptavidin plates (MSD L45SSA-1) were coated with 25 μl (or 15 μl for 384 well plates) of capture antibody diluted in Diluent 100 (MSD, R50AA-2) for 1 hour at room temperature with 700 rpm shaking (1000 rpm for 384-well). After TBST wash (3×), 25 μl samples were added each well (10 μl for 384-well) and incubated at 4° C. overnight with agitation at 700 rpm. After TBST wash (3×), 25 μl of detection antibodies (15 μl for 384-well) were added each well diluted in TBST containing 25% MSD blocker A (MSD R93AA-1), together with rabbit (Rockland Antibodies D610-1000) and mouse gamma globin fraction (D609-0100). After 1 hour incubation at room temperature at 700 rpm, followed by TBST washes (3×), 150 μl MSD read buffer (MSD R92TC. 1:1 diluted with water) is added (35 μl for 384-well), and plates are read on the MSD Sector S 600.

TABLE 1
Antibodies used in MSD assay.

| | Antibody | | | | Concentration |
|---|---|---|---|---|---|
| Assay | type | targets | Vendor | Cat No. | (μg/mL) |
| pS935 LRRK2 | Capture | pS935 LRRK2 | Abcam | ab133450 | 0.5 |
| | Detection | Total LRRK2 | BioLegend | 808201 | 1 |
| pT73 Rab10 | Capture | pT73 Rab10 | Denali | 19-4 | 1 |
| | Detection | Total Rab10 | Abcam | ab181367 | 2 |

FIG. 4 shows that at trough (predose) at steady state (Day 10 for Cohort B, Day 28 for Cohort D, Day 14 for Cohort E), compound I treatment resulted in a robust reduction in pS935 of ≥80% at the highest dose and ≥50% reduction at the lowest clinically relevant dose compared with baseline.

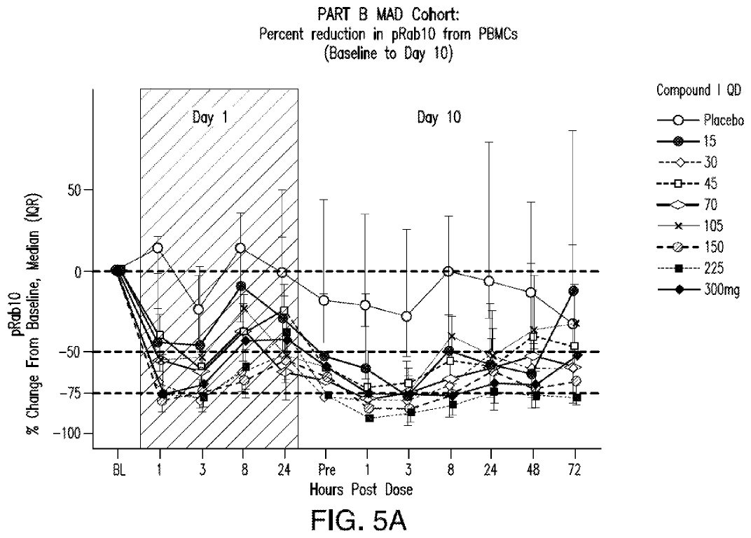

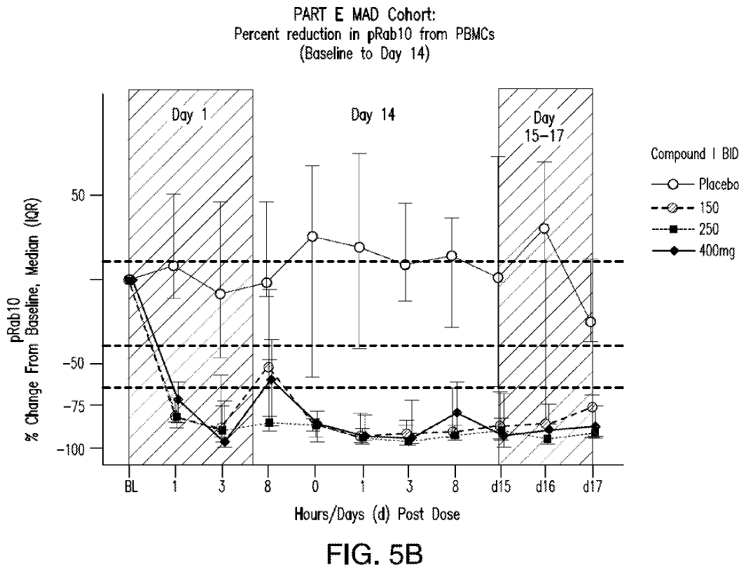

FIG. 5 shows that at trough (predose), compound I reduced phosphorylation of Rab10 (pRab10) in peripheral mononuclear cells (PBMCs), a direct substrate of LRRK2 kinase, in HVs by ≥70% at the highest dose at steady state (Day 10 for Cohort B, Day 28 for Cohort D, Day 14 for Cohort E).

Safety and Tolerability in Healthy Subjects after Multiple-Doses in Part D

Part D was composed of a multiple-dose cohort (Cohort D1) of healthy subjects administered either compound I 225 mg (n=13) or placebo (n=4) QD for 28 days.

In Part D, Compound I was generally well tolerated at a dose of 225 mg QD for 28 days in healthy subjects. No deaths, other SAEs, or AESIs were reported. One subject in the placebo group experienced a TEAE after the seventh dose, leading to study drug discontinuation (transaminases increased; considered moderate in severity and not related to study drug by the investigator). One subject withdrew consent after the eighth dose for personal reasons and was replaced. A total of 10 (77%) subjects in the compound I 225-mg QD group and 2 (50%) subjects in the placebo group experienced ≥1 TEAE (FIG. 9). The most common TEAE among compound I-treated subjects we headache (3 [23%] subjects) and procedure-related (3 [23%] subjects). Among the compound I-treated subjects, 10 (77%) experienced ≥1 mild TEAE and no subjects experienced a moderate or severe TEAE.

No other clinically significant changes in safety laboratory, vital sign, ECG, neurological examination, PFT, or C-SSRS results were observed.

Safety in Healthy Subjects after Multiple-Doses in Part E

Part E is composed of sequential MAD cohorts. Healthy subjects in Cohort E1 were administered compound I 150 mg or placebo BID (4:1 ratio) for 14 days (n=9) and subjects in Cohort E2 were administered compound I 250 mg or placebo BID (4:1 ratio) for 14 days (n=11). Subjects in the ongoing Cohort E3 are being administered compound 1400 mg or placebo BID (4:1 ratio) for 14 days. Unblinded safety data for Cohorts E1 and E2 are summarized below.

Based on unblinded safety data from Cohorts E1 and E2, compound I was generally well tolerated at 150 and 250 mg BID for 14 days in healthy subjects. No SAEs or AESIs were reported. Two study-drug related discontinuations occurred in Part E: one subject with moderate nausea, headache, impaired concentration, and diarrhea (250 mg BID); a second subject with severe headache and malaise with moderate nauseas (400 mg). One subject (250 mg BID) prematurely discontinued the study, the subject withdrew consent on Day 3 as a result of moderate TEAEs of nausea, headache, disturbance in attention, and diarrhea, all of which resolved on the day of onset after treatment with acetaminophen and were considered related to study drug by the investigator.

All 25 (100.0%) compound I-treated subjects and 6 (100.0%) placebo-treated subjects experienced 1 TEAE in the study. The most common TEAE among compound 1-treated subjects was headache (21 [84%] subjects vs. 4 [67%] subjects for placebo). The majority of TEAEs reported were mild or moderate in severity. One subject (250 mg BID) experienced a severe TEAE (procedural headache; considered not related to study drug by the investigator). Moderate TEAEs were reported for 2 subjects who received compound I 250 mg BID: headache (n=1) and headache, nausea, disturbance in attention, and diarrhea (n=1). Severe TEAEs were reported in two subjects: Procedural headache (n=1; 250 mg BID); Headache and malaise (n=1; 40 mg BID)

No clinically significant changes in vital sign, ECG, telemetry, safety laboratory (including liver and renal function testing), PFT, neurological examination, or the C-SSRS results were observed.

### Example 4: Phase 1b, Safety, Tolerability, Pharmacokinetic, and Pharmacodynamic Study in Subjects with Parkinson's Disease

### Demographics of Participants

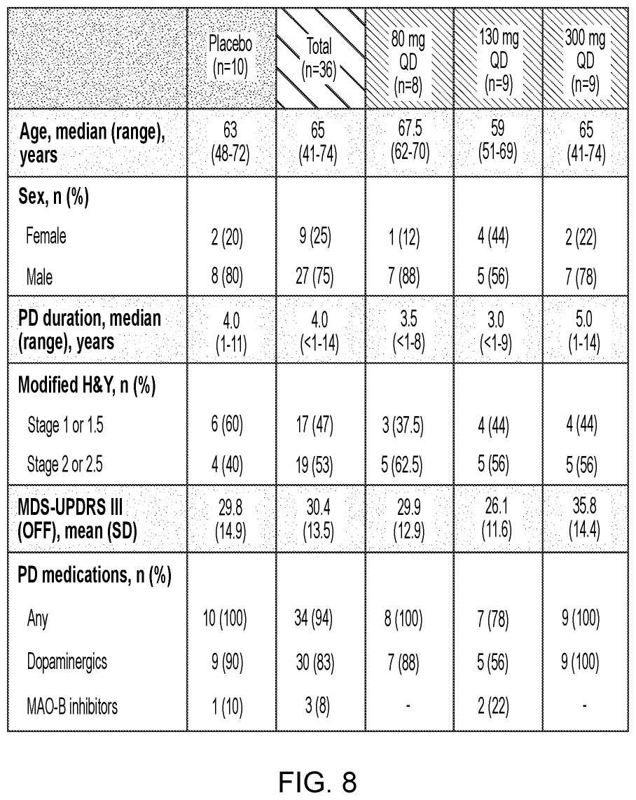

A total of 36 patients with PD (26 active, 10 placebo) were treated with up to 300 mg QD for 28 days (FIG. 8).

This was a Phase 1b, randomized, placebo-controlled, double-blind study designed to determine the safety, tolerability, PK, and pharmacodynamics of compound I in subjects with PD. The conduct of this study has been completed and the clinical study report is in progress. In Part 1, subjects were administered compound I 80 mg or placebo QD (1:1 ratio) for 28 days (n=8). In Part 2, subjects were administered compound I 80 or 130 mg or placebo QD (1:2:1 ratio) for 28 days (n=17). In Part 3, subjects were administered compound I 300 mg or placebo QD (4:1 ratio) for 28 days (n=11).

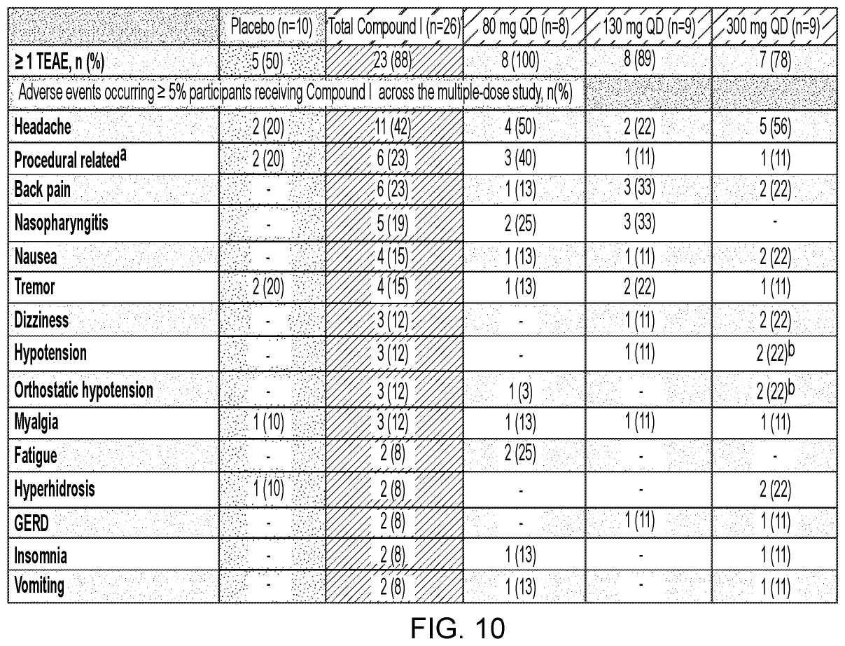

Based on unblinded safety data, compound I was generally well tolerated at 80, 130, or 300 mg QD for 28 days in subjects with PD (summarized in FIG. 10). No SAEs or AESIs were reported. Two (5.6%) subjects were discontinued from the study due a TEAE of hypotension after the first dose. For one of these subjects (130 mg QD), the event was asymptomatic and was considered severe and not related to study drug by the investigator. This subject was taking tamsulosin until one day prior to the event and had documented history of autonomic dysregulation. For the second subject (300 mg QD), the event was mildly symptomatic when standing and was considered mild and related to study drug by the investigator. Two additional subjects experienced TEAEs of mild orthostatic hypotension (80 mg QD) and mild hypotension plus orthostatic hypotension (300 mg QD), which resolved while on study drug.

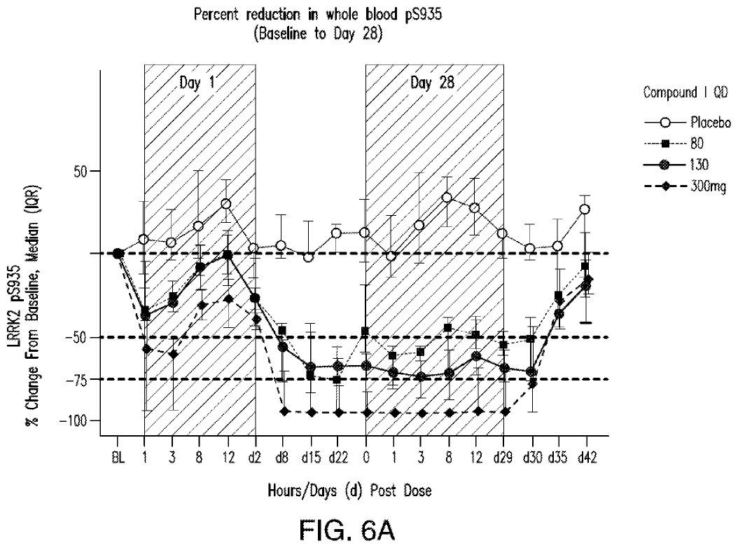

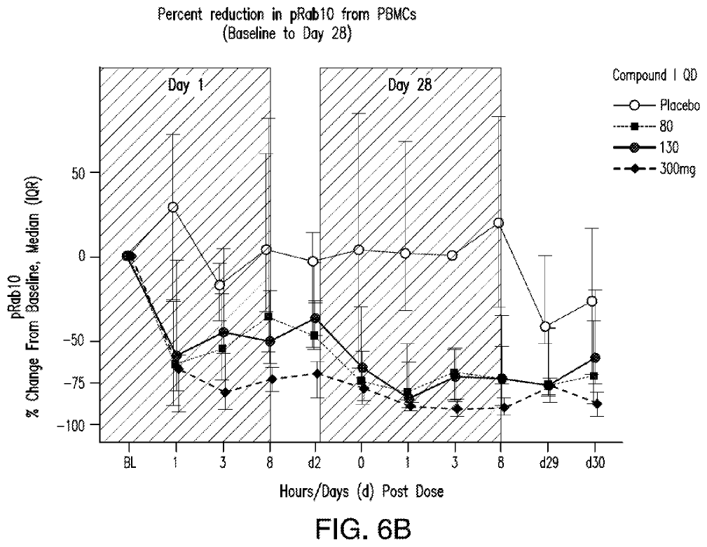

FIG. 6 shows that at trough (predose), at steady state (Day 28), compound I treatment resulted in robust reduction of pS935 in whole blood across all dose levels studied. At trough (predose), compound I reduced pRab10 in PBMCs in patients with PD across all doses at steady state.

A total of 23 (88.5%) compound I treated subjects and 5 (50%) placebo-treated subjects experienced ≥1 TEAE in the study (FIG. 10). The most common TEAE among compound I-treated subjects was headache (11 [42%] subjects vs. 2 [20%] subjects for placebo). The majority of TEAEs were mild or moderate in severity. Two subjects experienced severe TEAEs: one subject with hypotension (130 mg QD; considered not related to study drug due to pre-existing orthostasis) and one subject with headache (300 mg QD: considered related to lumbar puncture procedure and not related to study drug). Moderate TEAEs were experienced by 5 (19%) compound I-treated subjects and 1 (10.0%) placebo-treated subject: parkinsonism (decline in Parkinson's symptoms) (80 mg QD: n=1), fungal skin infection (130 mg QD; n=1), nuchal rigidity (130 mg QD; n=1), headache (130 and 300 mg QD; n=2), myalgia (300 mg QD: n=1), and constipation (placebo QD; n=1).

No other clinically significant individual changes from baseline or notable trends in safety laboratory (including liver and renal function testing), ECG, neurological assessment, vital sign, PFT, or C-SSRS results were observed.

Dose selection for further studies is based on the population PK/pharmacodynamic relationship of compound I plasma concentrations and validated target engagement biomarkers at exposures that were demonstrated to be safe and well tolerated.

### Conclusions

Safety: Compound I was generally well tolerated across a broad range of doses in HVs and patients with PD for up to 28 days. The most common TEAE in compound I-treated participants was headache. There were no clinically meaningful changes in pulmonary or renal function.

Pharmacokinetics: Compound I demonstrated high CSF penetration based on the CSF/unbound plasma ratio. Treatment with compound I achieved robust target engagement and pathway engagement at doses that were generally well tolerated in HVs and patients with PD.

Pharmacodynamics: Robust target and pathway engagement was achieved with compound I treatment in both HVs and patients with PD. In addition, compound I treatment in both HVs and patients with PD resulted in a dose-dependent reduction in the lysosomal lipid BMP 22:6 in urine, a marker of lysosomal function.

To date, no dose-dependent important safety concerns have been observed, with compound I found to be safe and generally well tolerated in over 200 subjects at doses up to 400 mg twice daily (BID) for 14 days for healthy volunteers or for a dose of 225 mg for 28 days.

### Example 5: Pharmaceutical Composition

An immediate release tablet formation was prepared containing 75 mg compound I mixed with the components shown in the table below in a dry granulation process.

TABLE 2

| Ingredients | % weight/weight |
|---|---|
| Compound I (75 mg) | 20 |
| Intragranular | |
| Microcrystalline cellulose | 67.5 |
| Povidone | 3 |
| Croscarmellose sodium | 2.5 |
| Talc | 1 |
| Colloidal silicon dioxide | 0.5 |
| Magnesium stearate | 0.5 |
| Extragranular | |
| Croscarmellose sodium | 2.5 |
| Talc | 1 |
| Colloidal silicon dioxide | 0.5 |
| Magnesium stearate | 1 |
| Coating | |
| Opadry ® White 03F580030 | 3 |

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

The foregoing invention has been described in some detail by way of illustration and example for purposes of clarity of understanding, the descriptions and examples should not be construed as limiting the scope of the invention. Accordingly, all suitable modifications and equivalents may be considered to fall within the scope of the invention as defined by the claims that follow.

What is claimed is:

1. A method for treating Parkinson's disease, the method comprising administering to a subject in need thereof between about 70 to 800 mg/day of compound I: 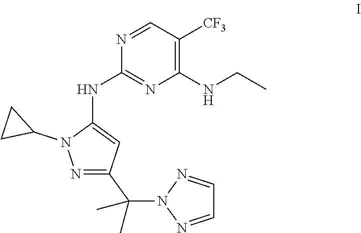 [SMILES: CCNc1nc(Nc2cc(C(C)(C)n3nccn3)nn2C2CC2)ncc1C(F)(F)F]
or a pharmaceutically acceptable salt or deuterated analog thereof.

2. The method of claim 1, wherein between about 70 to 225 mg of the compound is administered to the subject.

3. The method of claim 1, wherein between about 70 and 80 mg of the compound is administered to the subject.

4. The method of claim 1, wherein about 70 mg of the compound is administered to the subject.

5. The method of claim 1, wherein about 75 mg of the compound is administered to the subject.

6. The method of claim 1, wherein about 80 mg of the compound is administered to the subject.

7. The method of claim 1, wherein about 105 mg of the compound is administered to the subject.

8. The method of claim 1, wherein about 130 mg of the compound is administered to the subject.

9. The method of claim 1, wherein about 150 mg of the compound is administered to the subject.

10. The method of claim 1, wherein about 225 mg of the compound is administered to the subject.

11. The method of claim 1, wherein about 250 mg of the compound is administered to the subject.

12. The method of claim 1, wherein about 300 mg of the compound is administered to the subject.

13. The method of claim 1, wherein about 400 mg of the compound is administered to the subject.

14. The method of any preceding claim, wherein the compound is administered orally.

15. The method of any preceding claim, wherein the compound is administered once daily.

16. The method of any preceding claim, wherein the compound is administered twice daily.

17. The method of any preceding claim, wherein the method results in a reduction in phosphorylated S935 LRRK2 (pS935) in whole blood of the subject.

18. The method of any preceding claim, wherein the method results in a reduction in phosphorylated ras-related protein Rab10 (pRab10) in peripheral blood mononuclear cells (PBMC) of the subject.

19. The method of any preceding claim, wherein the method results in a reduction of lysosomal lipid 22:6-bis[monoacylglycerol]phosphate (BMP) in urine of the subject.

20. A method for treating Parkinson's disease, the method comprising administering once a day to a subject in need thereof between about 75 to 225 mg of compound I:

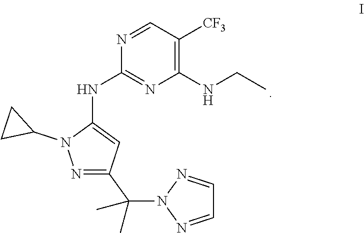 [SMILES: CCNc1nc(Nc2cc(C(C)(C)n3nccn3)nn2C2CC2)ncc1C(F)(F)F]

21. The method of claim 20, wherein about 75 mg of the compound is administered to the subject.

22. The method of claim 20 or 21, wherein about 150 mg of the compound is administered to the subject.

23. The method of any one of claim 20-22, wherein about 225 mg of the compound is administered to the subject.

24. A method for reducing phosphorylated S935 LRRK2 (pS935) in whole blood of a subject suffering from Parkinson's disease, the method comprising administering to a subject in need thereof between about 70 to 800 mg/day of compound I: 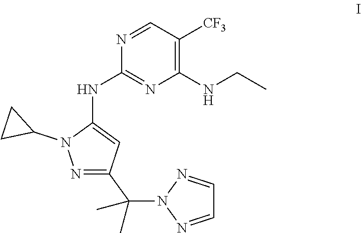 [SMILES: CCNc1nc(Nc2cc(C(C)(C)n3nccn3)nn2C2CC2)ncc1C(F)(F)F]
or a pharmaceutically acceptable salt or deuterated analog thereof.

25. The method of claim 24, wherein the pS935 is reduced by 41-97%.

26. A method for reducing phosphorylated ras-related protein Rab10 (pRab10) in peripheral blood mononuclear cells (PBMC) of a subject suffering from Parkinson's disease, the method comprising administering to a subject in need thereof between about 70 to 800 mg/day of compound I: 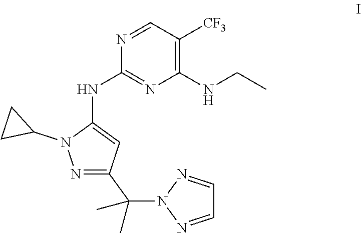 [SMILES: CCNc1nc(Nc2cc(C(C)(C)n3nccn3)nn2C2CC2)ncc1C(F)(F)F]
or a pharmaceutically acceptable salt or deuterated analog thereof.

27. The method of claim 26, wherein the pRab10 is reduced by 44-97%.

28. A method for reducing lysosomal lipid 22:6-bis[monoacylglycerol]phosphate (BMP) in urine of a subject suffering from Parkinson's disease, the method comprising administering to a subject in need thereof between about 70 to 800 mg/day of compound I: 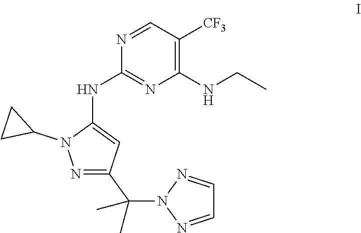 [SMILES: CCNc1nc(Nc2cc(C(C)(C)n3nccn3)nn2C2CC2)ncc1C(F)(F)F]
or a pharmaceutically acceptable salt or deuterated analog thereof.

29. The method of claim 28, wherein BMP(22:6/22:6)/or BMP(22:6/22:6)//creatinine is reduced by 22-86%.

30. The method of any preceding claim, wherein the subject is human.

31. The method of any preceding claim, wherein the Parkinson's disease is familial.

32. The method of any preceding claim, wherein the Parkinson's disease is sporadic.

33. Use of a LRRK2 inhibitor for treating Parkinson's disease, wherein the inhibitor is administered to a subject in need thereof between about 70 to 800 mg/day and is 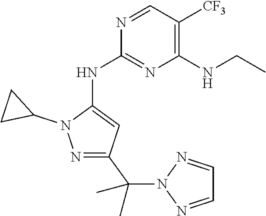 [SMILES: CCNc1nc(Nc2cc(C(C)(C)n3nccn3)nn2C2CC2)ncc1C(F)(F)F]
or a pharmaceutically acceptable salt or deuterated analog thereof.

34. Use of a LRRK2 inhibitor in the manufacture of a medicament for treating Parkinson's disease, wherein the inhibitor is administered to a subject in need thereof between about 70 to 800 mg/day and is 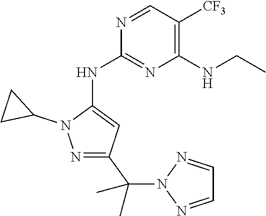 [SMILES: CCNc1nc(Nc2cc(C(C)(C)n3nccn3)nn2C2CC2)ncc1C(F)(F)F]
or a pharmaceutically acceptable salt or deuterated analog thereof.

35. A pharmaceutical composition comprising 70-800 mg of compound I, 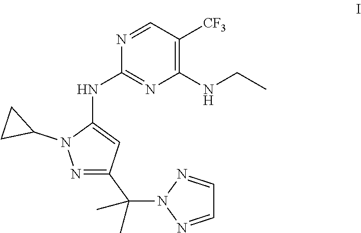 [SMILES: CCNc1nc(Nc2cc(C(C)(C)n3nccn3)nn2C2CC2)ncc1C(F)(F)F]
or a pharmaceutically acceptable salt or deuterated analog thereof and a pharmaceutically acceptable carrier.

36. The pharmaceutical composition of claim 35, comprising about 70-225 mg of compound I.

37. The pharmaceutical composition of claim 35, suitable for administration of about 800 mg per day.

38. The pharmaceutical composition of claim 35, suitable for administration of about 225 mg per day.

39. The pharmaceutical composition of claim 35, comprising about 70 mg of compound I.

40. The pharmaceutical composition of claim 35, comprising about 75 mg of compound I.

41. The pharmaceutical composition of claim 35, comprising about 80 mg of compound I.

42. The pharmaceutical composition of claim 35, comprising about 105 mg of compound I.

43. The pharmaceutical composition of claim 35, comprising about 130 mg of compound I.

44. The pharmaceutical composition of claim 35, comprising about 150 mg of compound I.

45. The pharmaceutical composition of claim 35, comprising about 225 mg of compound I.

46. The pharmaceutical composition of claim 35, comprising about 250 mg of compound I.

47. The pharmaceutical composition of claim 35, comprising about 300 mg of compound I.

48. The pharmaceutical composition of claim 35, comprising about 400 mg of compound I.

49. The pharmaceutical composition of claim 35, suitable for oral administration.

50. The pharmaceutical composition of claim 35, suitable for administration once daily.

51. The pharmaceutical composition of claim 35, suitable for administration twice daily.

52. The pharmaceutical composition of claim 35, suitable for administration three times daily.
